(19) United States
(12) Patent Application Publication
(10) Pub. No.: US 2016/0024478 A1
(43) Pub. Date: Jan. 28, 2016

(54) Active Phage-Based Inks, Methods of Printing on Materials and Phage-Based Bioactive

(71) Applicant: University of Guelph, Guelph (CA)

(72) Inventors: Mansel Griffiths, Rockwood (CA); Hany Anany, Guelph (CA); Lioubov Brovko, Guelph (CA)

(21) Appl. No.: 14/808,818

(22) Filed: Jul. 24, 2015

Related U.S. Application Data
(60) Provisional application No. 62/101,607, filed on Jan. 9, 2015.
(60) Provisional application No. 62/028,561, filed on Jul. 24, 2014.

(51) Int. Cl. C12N 7/00; C12Q 1/10; A01N 25/34; A01N 63/00; A01N 25/08

(52) U.S. Cl. CPC C12N 7/00; A01N 63/00; A01N 25/08; A01N 25/34; C12Q 1/10; C12N 2795/00031

(57) ABSTRACT

An active phage bio-ink composition and phage-based bioactive paper, wherein the phage is oriented and immobilized at the paper surface through heads, with the phage tail fibers free to capture target bacterial pathogens.

### CROSS REFERENCE TO EARLER APPLICATION

This application claims priority to U.S. provisional application Ser. No. 62/028,561 filed Jul. 24, 2014 and also to U.S. provisional application Ser. No. 62/101,607 filed Jan. 9, 2015, the contents of both of which are incorporated herein by reference in their entirety.

### FIELD OF THE INVENTION

The present invention relates to immobilized phage as a bioactive material for applications in research and industry. More specifically, the invention relates to active phage bio-ink compositions, packaging materials containing such compositions printed thereon, and methods of printing such compositions on materials to form phage-based bioactive packaging materials. The invention also encompasses methods to detect bacterial pathogens in samples using phage-based bioactive materials.

### BACKGROUND OF THE INVENTION

Microorganisms have been immobilized to a support matrix using techniques such as physical adsorption, gel entrapment and covalent binding (Knaebel et al., 1997, Selvaraj et al., 1997, Jirku, 1999) for applications in different areas, for instance in food biotechnology and in waste water treatment. Surface immobilization of microorganisms by adsorption has the advantage of being very simple to carry out and there is no transfer barrier as in the entrapment method (Klein and Ziehr, 1990). On the other hand, the relative interaction between the support and the cells is low resulting in the release of the immobilized material to the surrounding medium. Covalent binding between a functional group of the cells, generally amino acids, and support matrix has been reported as another method of immobilization (Jirku, 1999). This provided a strong binding and overcame the diffusion problem of simple adsorption technique, but some chemicals which might be used to create covalent cross-linking, such as glutaraldehyde, could result in the loss of the activity and viability of the cells.

The bacteriocin, pediocin (ALTA 2351) has been incorporated in cellulose matrix films, and these bioactive films were evaluated to control growth of Listeria innocua and Salmonella spp. and extend shelf life of artificially contaminated ready-to-eat ham slices (Santiago-Silva et al., 2009). Another bacteriocin, nisin, has been immobilized at different concentrations into palmitoylated alginate-based films or in activated alginate beads and the antimicrobial efficiency has been examined using artificially contaminated beef muscle slices and ground beef with Staphylococcus aureus and stored at 4° C. for 7 and 14 days respectively (Millette et al., 2007). Nisin has been also incorporated into a polyethylene-based plastic film and tested to inhibit surface growth of bacteria on meat (Siragusa et al., 1999).

Properties of phages in specifically interacting and lysing their host bacteria make them another bioactive material that can be used in the immobilized form to increase their application in research and industrial fields. US2012/0258175 discloses phages that have been absorbed onto a solid matrix (such as skim milk powder, soya protein powder and whey protein powder) and then dried by heating under vacuum. It was suggested that these immobilized phages may be used for phage therapy applications in human, veterinary and aquaculture in addition to agricultural applications.

Electrospinning has also been used for the encapsulation and immobilization of T7, T4, λ phages in electrospun polymer nanofibres (Salalha et al., 2006). The potential of nanoencapsulating of a broad lytic phage (φ-PVP-SE1) in water-in-oil-in-water (W/O/W) multiple emulsion has been also investigated (Costa et al., 2009). In a similar approach, phages against Staphylococcus aureus or Pseudomonas aeruginosa have been encapsulated into biodegradable polyester microspheres with only a partial loss of lytic activity after being frozen in liquid nitrogen and lyophilized for over 72 hours (Puapermpoonsiri et al., 2009). In all the above mentioned techniques, phages are enclosed in protective material and they need to be released to be able to infect target pathogens, so these techniques could be used in phage therapy applications where phages need to overcome some environmental barriers before reaching sites of the target pathogen.

Cocktails of E. coli 0157:H7 or Listeria monocytogenes phages immobilized on cellulose membranes have been shown to control the growth of their host in raw and ready-to-eat meats, respectively, under different storage temperatures and packaging conditions (Anany et al., 2011). Recently, attempts have been made to encapsulate different phages using different chemical formulations in alginate microspheres and gelatin capsules to provide protection for these phages against the low pH found in the stomach upon oral delivery (Jiayi et al., 2010, Ma et al., 2008, Stanford et al., 2010, Yongsheng et al., 2012).

Other studies have reported other techniques for phage immobilization and the potential use of the developed phage-based biosorbent as biosensors to detect, concentrate and identify target bacteria. Salmonella have been captured from mixed cultures using Salmonella-specific phage passively immobilized on polystyrene supports (Bennett et al., 1997). Chemical biotinylation of the phage head is another approach to immobilize phages where they were immobilized on the surface of streptavidin-coated magnetic beads (Sun, 2001).

Phages have been immobilized and employed as recognition receptors in a number of biosensors. Physical adsorption has been used to immobilize IG40 filamentous phages on gold surfaces of quartz crystal microbalance (QCM) biosensor and used as recognition elements to detect β-galactosidase from E. coli (Nanduri et al., 2007a). Filamentous phage Lm P4:A8, expressing the scFv antibody to virulence factor actin polymerization protein (ActA) on its surface, was immobilized to the surface plasmon resonance (SPR) sensor surface through physical adsorption and used for detection of Listeria monocytogenes (Nanduri et al., 2007b). Filamentous phage specific to Salmonella Typhimurium has been physically adsorbed to the surface of magnetoelastic sensor and used as a biorecognition element for detection of its host cells at different concentrations (Lakshmanan et al., 2007). Staphylococcus lytic phages were immobilized on the gold surface of surface plasmon resonance (SPR) sensor via direct physical adsorption (Balasubramanian et al., 2007).

Enzyme-linked immunosorbent assay and atomic force microscopy has been used to covalently immobilize Podoviridae Salmonella phage P22 to glass substrates (Handa et al., 2008). Moreover, wild type T4 tailed phage has been immobilized on modified gold surfaces of SPR sensor through hydrogen bonding (Singh et al., 2009). Electrochemically modified screen printed electrode (SPE) microarrays have been developed to covalently immobilize T4 phages and use these phages as recognition receptors for the detection of E. coli through impedance measurements (Shabani et al., 2008).

Site-specific immobilization of phages has been suggested to overcome the orientation problem of immobilized phages and as a consequence increase the capture efficiency to the target bacteria. Phage display technology, in which foreign gene fragments encoding a polypeptide are inserted into the phage genome through fusion with one of the coat protein genes in order to be expressed on the phage's surface, facilitates the production of recombinant phages that are able to display different proteins on their heads (Paschke, 2006).

It was reported that electrostatic interactions between the charged proteins and the charged membranes might play a role in the separation of proteins through ultrafiltration membranes (Bhushan and Etzel). Positively-charged ultrafiltration membranes substantially improved the separation of proteins that have a small difference in molecular weight (such as β-lactoglobulin and glycomacropeptide). The application of charged membranes may also be used to improve high-performance tangential flow filtration (HPTFF) for protein purification (van Reis et al., 1999).

The charge difference approach might prove a simple approach to enhance site-specific immobilization of different phages without going through the genetic modification protocol. For the T4 phage the isoelectric points (pKa) of capsid and tail fibers as single units have not been determined and as a result there is no available report for the overall charge at a given pH for these components. However it was reported that the net charge on most viruses is negative and the whole T4 phage (capside, tail and fibers) has an isoelectric point close to 4 (Archer and Liu, 2009). The pKa values for major T4 head proteins were found to have a range between 4.62 and 6.63, while a range between 5.21 and 9.76 was reported for tail fiber proteins (Cummings et al., 1970, Showe and Onorato, 1978, Karam et al., 1994). Based on these values, it was suggested that capsids acquire a negative overall charge above pH 4 (Archer and Liu, 2009). In an early study, T7 phage head was suggested to be primarily responsible for the overall negative charge of the phage and the tail fibers could be positively charged (Serwer and Hayes, 1982). In a recent study, it was found that T4 heads were aggregated at pH 5.6 and 7.5 on aminosilanized substrate, where capsids behave as negatively charged entities and electrostatically attracted to a positively charged surface (Archer and Liu, 2009). Lower aggregation of T4 phage heads occurred with slightly positive and negative surfaces due to weaker electrostatic interaction and repulsion forces that occurred respectively. This lower aggregation suggested that adsorption of T4 capsids might not be purely electrostatic, but rather a combination of various types of interactions. It was also reported that presence of divalent counterions could facilitate the interaction and overcome electrostatic repulsion (Archer and Liu, 2009) (Pastre et al., 2003).

It is apparent that it is desirable to immobilize phages within a variety of substrates using a variety of methods. Several of these methods are use specific and several of these methods are complex and time consuming with inconsistent results. Studies indicating that phages may have a net negative charge on their capsid (heads) and positive charge on their tails have now provided a mechanism for the development of oriented immobilization of phages in cationic materials using novel compositions with improved results.

### SUMMARY OF THE INVENTION

The present invention presents active phage bio-ink compositions and a novel method for printing such active phage bio-ink compositions onto a substrate to produce a phage-based bioactive substrate that can be used to detect and/or control a desired pathogen. The phage-based bioactive substrate can be utilized as a packaging material to control the growth of various foodborne pathogens on food (including but not limited to meats, produce, dairy products such as cheeses). This proposed approach can help to broaden phage application not only to enhance food safety but also in many fields such as phage therapy in humans and animals and in environmental control in public spaces and preventing cross-contamination by bacteria.

In aspects, the substrate is a cationic substrate since it is believed that phages may have a net negative charge on their capsids (heads) and positive charge on their tails. Thus cationic substrates are useful for oriented immobilization of the phages at the substrate aspects, the substrate is any printable cationic substrate. Non-limiting examples of suitable printable cationic substrates may include papers, films, casings and packaging materials.

Furthermore, the use of printing technology is a worthwhile approach to develop commercial applications of biomolecules and has now facilitated the development of phage-based bioactive paper as described herein.

According to an aspect of the present invention is a phage bio-ink composition. According to a further aspect of the present invention is a lytic phage bio-ink composition comprising:

one or more lytic active phages specific for food borne pathogenic bacteria;

surfactant; and

viscosity modifier.

According to a further aspect of the present invention is a lytic phage bio-ink composition comprising:

one or more lytic active phages specific for food borne pathogenic bacteria;

surfactant;

viscosity modifier;

optional polysaccharide stabilizer; and

optional pigment.

According to a further aspect of the present invention is a lytic phage bio-ink composition comprising:

one or more lytic active phages specific for food borne pathogenic bacteria;

surfactant;

viscosity modifier;

optional pigment.

According to a further aspect of the present invention is a lytic phage bio-ink composition comprising:

one or more lytic active phages specific for one or more bacteria, which may include but is not restricted to Campylobacter jejuni, Cronobacter spp., shiga-toxin producing E. coli (STEC), Listeria monocytogenes, Salmonella spp., Vibrio spp. and Shigella spp.

surfactant; and

viscosity modifier.

According to a further aspect of the invention is a method to make a phage-based bioactive paper, the method comprising:

printing a lytic active phage bio-ink composition comprising one or more lytic phages specific for food borne pathogenic bacteria, surfactant ,viscosity modifier and optional polysaccharide onto a cationic paper; wherein said printing is done by an ink jet printer that does not employ heat;

drying the printed cationic paper; and

storing the printed cationic paper at a relative humidity of about 80%.

In aspects the printing is piezoelectric printing.

According to a further aspect of the invention is a method to make a phage-based bioactive paper, the method comprising:

printing a lytic active phage bio-ink composition comprising one or more lytic phages specific for food borne pathogenic bacteria, surfactant and viscosity modifier onto a cationic paper; wherein said printing is done by a piezoelectric ink jet printer,

drying the printed cationic paper; and

storing the printed cationic paper at a relative humidity of about 80% to about 85%.

According to a further aspect of the invention is a method to make a phage-based bioactive paper, the method comprising:

printing a lytic active phage bio-ink composition comprising one or more lytic phages specific for food borne pathogenic bacteria, surfactant and viscosity modifier onto a cationic paper; wherein said printing is done by a piezoelectric ink jet printer,

drying the printed cationic paper and maintaining the printed cationic paper at a relative humidity of about 80% or more, wherein about 10⁶ PFU of phage is provided per cm² of paper.

A phage-based bioactive dipstick (test strip) for use in methods to detect target bacterial pathogens in a sample, the dipstick comprising a phage-based bioactive paper, wherein the phage is oriented and immobilized at the paper surface through heads, with the phage tail fibers free to capture the target bacterial pathogens. In aspects, about 10⁶ PFU of phage is provided per cm² of paper.

A phage-based bioactive packaging material for packing foods, the packaging material comprising a phage-based bioactive paper, wherein the phage is oriented and immobilized at the paper surface through heads, with the phage tail fibers free to capture the target bacterial pathogens, wherein about 10⁶ PFU of phage is provided per cm² of paper.

A phage-based bioactive paper, wherein the phage is oriented and immobilized at the paper surface through heads, with the phage tail fibers free to capture the target bacterial pathogens, wherein about 10⁶ PFU of phage is provided per cm² of paper.

A food-packaging paper capable of inhibiting growth of a target bacterium in food during storage, the paper comprising phage that is oriented and immobilized at the paper surface through heads, with the phage tail fibers free to capture the target bacterial pathogens, wherein about 10⁶ PFU of phage is provided per cm² of paper.

According to a further aspect of the invention is a method to detect a bacterial pathogen in a sample, the method comprising:

(a) providing a dipstick comprising a phage-based bioactive paper, wherein the phage is oriented and immobilized at the paper surface through heads, with the phage tail fibers free to capture the target bacterial pathogens;

(b) dipping (a) into the sample to bind said bacterial pathogen;

(c) transferring (b) into a clean incubation broth and incubating;

(d) subjecting (c) to a detection method to detect progeny phage released from the bacterial pathogen.

In aspects, the method is done in tandem with a control that contains no bacterial pathogens. In further aspects, the method can detect as low as 10 cfu/g or less via enumerating progeny phage released from the pathogenic bacterium.

According to a further aspect of the invention is a method to detect a spoilage or pathogenic bacteria in a sample, the method comprising:

(a) providing a dipstick comprising a phage-based bioactive paper, wherein the phage is oriented and immobilized at the paper surface through heads, with the phage tail fibers free to capture the target the spoilage or pathogenic bacteria;

(b) dipping (a) into the sample to bind said spoilage or pathogenic bacteria;

(c) transferring (b) into a clean incubation broth and incubating;

(d) subjecting (c) to a detection method to detect progeny phage released from the spoilage or pathogenic bacteria.

The foregoing has broadly outlined the features and technical advantages of the present invention in order that the detailed description of the invention that follows may be better understood. Additional features and advantages of the invention will be described hereinafter which form the subject of the claims of the invention. It should be appreciated by those skilled in the art that the specific embodiment disclosed herein may be readily utilized as a basis for modifying or designing other structures for carrying out the same purposes of the present invention. It should also be realized by those skilled in the art that such equivalent constructions do not depart from the scope of the invention. The novel features which are believed to be characteristic of the invention, both as to its organization and method of operation, together with further objects and advantages will be better understood from the following description when considered in connection with the accompanying figures. It is to be expressly understood, however, that each of the figures is provided for the purpose of illustration and description only and is not intended as a definition of the limits of the present invention.

### BRIEF DESCRIPTION OF THE DRAWINGS

FIG. 1 is a transmission electron micrograph for E. coli 045 phage;

FIG. 2 is a transmission electron micrograph for the printed phage Salmonella Newport phage (CGG 4-1 phage);

FIG. 3 shows the effect of printed phage on Listeria monocytogenes;

FIG. 4 is a diagram of E. coli 0157:H7 detection protocol with phage-paper of the present invention;

FIG. 5; is the methodology used for phage-printed paper strips in medium to detect bacteria;

FIG. 6 is the dipstick methodology used for phage-printed paper strips to detect bacteria;

FIG. 7 are the detection results of E. coli 0157:H7 in broth using the spotting technique;

FIG. 8 are the detection results of E. coli 0157:H7 in broth (qPCR);

FIG. 9 are the detection results of E. coli 0157:H7 in spinach homogenates (qPCR);

FIG. 10 are the detection results of E. coli 0157:H7 in spinach homogenates (spotting);

FIG. 11 shows the dip detection results of E. coli 0157:H7 in broth (spotting);

FIG. 12 shows the dip detection results of E. coli 0157:H7 in broth (qPCR);

FIG. 13 shows the dip detection results of E. coli 0157:H7 from spotting from spinach samples;

FIG. 14 shows the dip detection results with E. coli 0157:H7 from qPCR from spinach samples;

FIG. 15 shows the infectivity of immobilized phage on E. coli ;

FIG. 16 shows the infectivity of immobilized 4-1 phage on Salmonella Newport and its stability after one week;

FIG. 17 shows the infectivity of the gravure printed and blade coated AG11 phage-based paper determined by the overlay method after 24 h incubation at 25° C. A: Gravure printed AG11, B: Blade coated AG11. The diameters of zones of lysis were measured to determine the infectivity of bioactive paper;

FIG. 18 shows the infectivity of the gravure printed bioactive paper in broth. Bioactive paper was added to TSB culture of 10³ cfu/ml of respective host bacteria and incubated for 18-24 h at 25° C. Bacterial counts after incubation with bioactive paper were compared to a control containing paper without phage;

FIG. 19 shows the effect of bio-ink containing 30% glycerol for piezoelectric inkjet printing on phage titre. Phages were resuspended in bio-inks and the titre was determined initially and after 1, 2 and 5 h;

FIG. 20 shows the effect of bio-ink containing 50% glycerol for piezoelectric inkjet printing on phage titre. Phages were resuspended in bio-inks and the titre was determined initially and after 1, 2 and 5 h;

FIG. 21 shows the effect of piezoelectric printing on phages. Phage bio-ink solutions were printed for approximately 15 min directly into a Petri dish. The phage titres were determined before and after printing;

FIG. 22 shows the infectivity of phage-based Whatman paper in broth culture. Phages were printed on Whatman no. 1 paper by thermal and piezoelectric inkjet printing. Papers were added to broth cultures containing 10³ cfu/ml of respective host bacteria and incubated for 18-24 h to determine the efficacy of each phage-based paper;

FIG. 23 shows the infectivity of AG6 phage-based paper in broth culture. AG6 phage was printed on Whatman paper and cationic-coated papers (PVAm, Hydrogel, ColorLok) by piezoelectric inkjet printing. Paper discs were added to broth cultures containing 10³ cfu/ml of S. Typhimurium and incubated for 18-24 h to determine the biocontrol effect of each phage-based paper;

FIG. 24 shows the infectivity of AG10 phage-based paper in broth culture. AG10 phage was printed on Whatman paper and cationic-coated papers (PVAm, Hydrogel, ColorLok) by piezoelectric inkjet printing. Paper discs were added to broth cultures containing 10³ cfu/ml of E. coli 0157:H7 and incubated for 18-24 h to determine the biocontrol effect of each phage-based paper;

FIG. 25 shows the infectivity of AG11 phage-based paper in broth culture. AG11 phage was printed on Whatman paper and cationic-coated papers (PVAm, Hydrogel, ColorLok) by piezoelectric inkjet printing. Paper discs were added to broth cultures containing 10³ cfu/ml of S. Enteritidis and incubated for 18-24 h to determine the biocontrol effect of each phage-based paper;

FIG. 26 shows the infectivity of AG20 phage-based paper in broth culture. AG20 phage was printed on Whatman paper and cationic-coated papers (PVAm, Hydrogel, ColorLok) by piezoelectric inkjet printing. Paper discs were added to broth cultures containing 10³ cfu/ml of L. monocytogenes and incubated for 18-24 h to determine the biocontrol effect of each phage-based paper;

FIG. 27 shows the infectivity of printed ColorLok phage-based paper in broth culture. Phages AG6, AG10, AG11, AG20 were printed on ColorLok paper by piezoelectric inkjet printing. Paper discs were added to broth cultures containing 10³ cfu/ml of S. Typhimurium, E. coli 0157:H7, S. Enteritidis or L. monocytogenes and incubated for 18-24 h to determine the biocontrol effect of each phage-based paper;

FIG. 28 shows the infectivity of pipetted ColorLok phage-based paper in broth culture. Phages AG6, AG10, AG11, AG20 were pipetted on ColorLok paper. Paper discs were added to broth cultures containing 10³ cfu/ml of S. Typhimurium, E. coli 0157:H7, S. Enteritidis or L. monocytogenes and incubated for 18-24 h to determine the biocontrol effect of each phage-based paper;

FIG. 29 shows the infectivity of stored ColorLok printed phage-based paper in broth culture. Phages AG6, AG10, AG11, AG20 were printed on ColorLok paper by piezoelectric inkjet printing. Phage-based paper was stored for 1 month at room temperature at 80-85%

RH. Papers were then added to broth cultures containing 10³ cfu/ml of S. Typhimurium, E. coli 0157:H7, S. Enteritidis or L. monocytogenes and incubated for 18-24 h to determine the biocontrol effect of each phage-based paper;

FIG. 30 shows the infectivity of stored ColorLok pipetted phage-based paper in broth culture. Phages AG6, AG10, AG11, AG20 were pipetted on ColorLok paper then phage-based paper was stored for 1 month at room temperature at 80-85% RH. Papers were then added to broth cultures containing 10³ cfu/ml of S. Typhimurium, E. coli 0157:H7, S. Enteritidis or L. monocytogenes and incubated for 18-24 h to determine the biocontrol effect of each phage-based paper;

FIG. 31 shows the effect of different concentrations of free AG6 phage on counts of S. Typhimurium (initial inoculum level of 10³cfu/m1) after 18-24 h at 25° C.;

FIG. 32 shows the effect of different concentrations of free AG10 phage on counts of E. coli 0157:H7 (initial inoculum level of 10³ cfu/ml) after 18-24 h at 25° C.;

FIG. 33 shows the effect of different concentrations of free AG11 phage on counts of S. Enteritidis (initial inoculum level of 10³cfu/m1) after 18-24 h at 25° C.;

FIG. 34 shows effect of different concentrations of free AG20 phage on counts of L. monocytogenes (initial inoculum level of 10³ cfu/m1) after 18-24 h at 25° C.;

FIG. 35 shows the methodology used for the phage printed paper and phage-free paper strips with salmonella bacteria;

FIG. 36 shows the inhibition of Salmonella Newport growth using immobilized phage (A): Bacteria+phage-printed bioactive paper+TSB, (B): Bacteria+TSB after incubation for 18hr. at 22° C.;

FIG. 37 shows the infectivity and stability of phage-based bioactive paper on Salmonella Newport. Phage-printed paper, paper without phage (printed using ink only), phage suspension (around 1.00E+06 PFU/mL) were added to around 1.00E+03 CFU/mL Salmonella Newport and incubated at 25° C. The bacterial count was determined after 18 hrs. The whole experiment was done after storing the phage-printed paper for 1 day, 1 week and 2 weeks at room temperature at approximately 80% relative humidity (RH). Data are mean of three replicates;

FIG. 38 shows the effect of ink composition on the activity of Salmonella Newport phage. The Salmonella Newport phage (CGG 4-1) suspension was mixed with 2mM Triton X 100, 30% Glycerol and stored for 1 day, 1 week and 1 month at room temperature. The phage count (PFU/mL) was determined after storage period using spot test technique. Data are from the mean of three replicates;

FIG. 39 shows the methodology for the dipstick approach for phage-printed paper and phage-free paper strips to 1 mL bacteria sample, where the bacteria is salmonella;

FIG. 40 shows Detection of Salmonella Newport in broth (TSB) using phage-printed paper (Dipstick approach). The progeny phages were counted (PFU/ml) using overlay technique. Data are from the mean of three replicates. Detection limit is around 10 CFU/mL. The whole test was done within around 24 hours (5.5 hrs incubation of phage with the bacteria+18.5 hrs to count phage using overlay technique);

FIG. 41 shows the detection of Salmonella Newport in broth (TSB) using phage-printed paper (Dipstick approach). The progeny phages were detected using RTPCR (Ct values). Data are from the mean of three replicates. Detection limit is around 10 CFU/mL. The whole test was done within around 8 hours (5.5 hrs incubation of phage with the bacteria+2 hrs to detect the p Detection of Salmonella Newport in broth (TSB) using phage-printed paper (Dipstick approach). The progeny phages were counted (PFU/ml) using overlay technique. Data are from the mean of three replicates. Detection limit is around 10 CFU/mL. The whole test was done within around 24 hours (5.5 hrs incubation of phage with the bacteria+18.5 hrs to count phage using overlay technique to detect the phages using RTPCR);

FIG. 42 shows the detection of Salmonella Newport in broth (TSB) using phage-printed paper stored for one week at room temperature with approximately 80% RH. The progeny phages were counted (PFU/ml) using overlay technique. Data are from the mean of three replicates. Detection limit is around 10 CFU/mL. The whole test was done within around 24 hours (5.5 hrs incubation of phage with the bacteria+18.5 hrs to count phage using overlay technique);

FIG. 43 shows the detection of Salmonella Newport in broth (TSB) using phage-printed paper stored for one week at room temperature with approximately 80% RH. The progeny phages were detected using RTPCR (Ct values). Data are from the mean of three replicates. Detection limit is around 10 CFU/mL. The whole test was done within around 8 hours (5.5 hrs incubation of phage with the bacteria+2 hrs to detect the phages using

RTPCR);

FIG. 44 shows the dipstick approach using chicken as a sample in order to detect salmonella using the phage-paper of the invention;

FIG. 45 shows the detection of Salmonella Newport in chicken using phage-printed paper (Dipstick approach). The progeny phages were counted (PFU/mL) using overlay technique. Data are from the mean of three replicates. Detection limit is around 50 CFU/mL. The whole test was done within around 24 hours (5.5 hrs incubation of phage with the bacteria +18.5 hrs to count phage using overlay technique); and

FIG. 46 shows the detection of Salmonella Newport in chicken using phage-printed paper (Dipstick approach). The progeny phages were detected using RTPCR (Ct values). Data are from the mean of three replicates. Detection limit is around 50 CFU/mL. The whole test was done within around 8 hours (5.5 hrs incubation of phage with the bacteria+2 hrs to detect the phages using RTPCR).

### DETAILED DESCRIPTION OF THE INVENTION

The invention provides an easy-to-use, inexpensive, sensitive and rapid method to detect bacteria, such as foodborne bacterial pathogens and spoilage bacteria in foods within a working day without the need for enrichment. This is desirable and necessary to provide the sensitivity needed by the food industry to demonstrate regulatory compliance and also helps to decrease potential contamination of the food production environment. With the present invention also is the provision of a method to control and/or reduce pathogenic bacteria in the food industry.

The invention also provides active phage bio-ink compositions used to prepare test strips (dip sticks) with printed oriented and active phage to test for the presence of a host bacteria in a sample that are killed by the phage and, therefore cannot be disseminated. Thus the test strips of the invention can be used in methods to detect progeny phage released from target bacterium. The active phage bio-ink compositions are also for use generally to provide phage-based bioactive paper with the phages oriented such that the head of the phage is at the paper surface and the tail of the phase is free to capture a bacterium. Such paper can be used in a wide variety of applications including as packaging material to control the growth of various foodborne bacterial pathogens and spoilage bacteria.

The current application of phages in the food industry is limited to the use of liquid phage preparations for bio-control purposes, where immobilized phage tail fibers or receptor binding proteins are used to specifically capture the target bacteria. Development of active phage-based bioactive substrates/packaging is an economically feasible approach for commercial applications of phages in food and generally in the food industry. This helps to ensure that phages are applied and retained near to the surface that is being treated, thereby avoiding excessive phage wastage.

Immobilized, oriented phages as in the present invention are whole phage particles and can be used to capture and infect target bacteria and the released progeny phages can be detected using different immunological or molecular based techniques, such as RT-PCR, qPCR, isothermal nucleic acid amplification techniques, and ELISA-based techniques. This phage-based detection approach has an enhanced sensitivity, selectivity and reduced detection time when compared with other available detection techniques.

An important aspect of the invention is that only viable and metabolically active bacterial cells are detected, which is not the case for many molecular-based detection technologies. Another important aspect of the present invention is that the initial oriented and active phage printed onto a test strip is not substantially released into the sample being tested for bacteria. Thus only the progeny phages are detected in methods using the printed test strip of the present invention which leads to more accurate and sensitive detection of bacteria. The invention in aspects also provides the possibility of creating packaging materials that target specific bacterial pathogens. Most foodborne illness outbreaks are caused by specific pathogen/food combinations, for example, Listeriosis from ready to eat deli meats and soft cheeses, so it will be possible to design and fabricate packaging material to increase the safety of foods by targeting the pathogens most closely associated with them.

The invention in aspects provides for an active phage bio-ink composition that comprises one or more active phages, surfactants and viscosity modifiers. The phage for use in the compositions of the invention that are printed onto a material such as paper, can be any type of active phage specific for a foodborne bacterial pathogen or spoilage bacteria. In aspects, the active phages are lytic bacteriophages (phages) that possess antibacterial properties by infecting host specific bacteria, which results in bacterial cell lysis and the release of progeny phages. This makes them an ideal bioactive material that can be used in the immobilized form to increase their application in food, research and industrial fields. In aspects, lytic phages from different phage families such as Leviviridae, Siphoviridae and Myoviridae families are suitable.

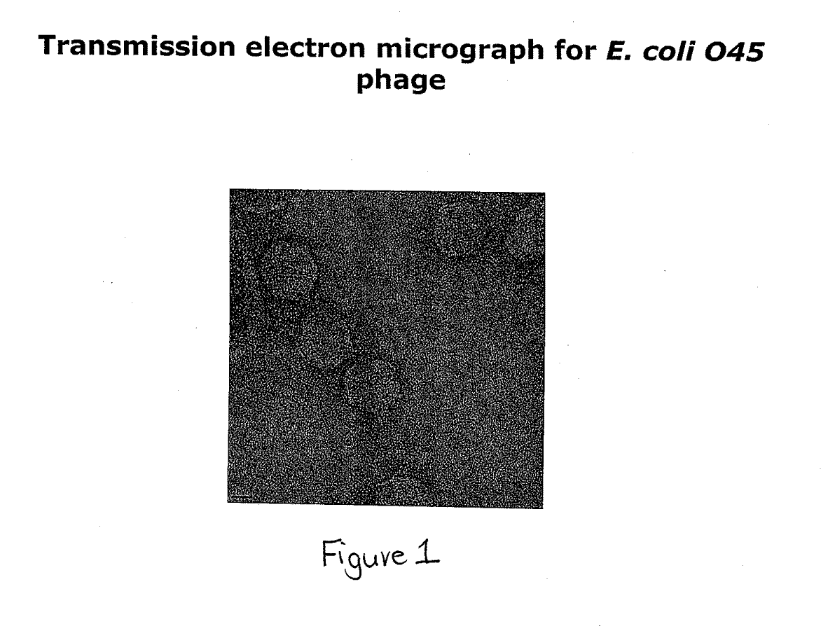

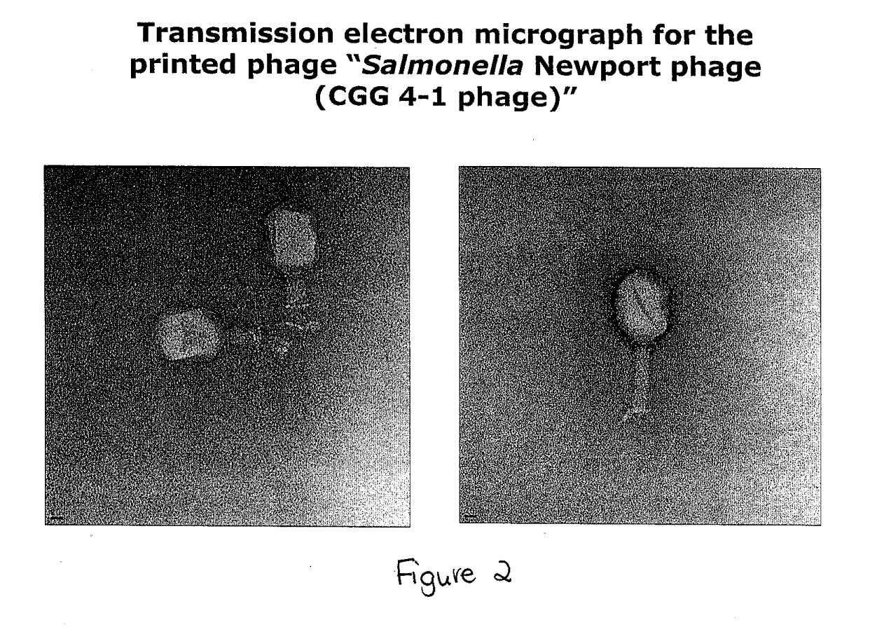

Suitable lytic active phages for use in the present invention may include but not be limited to rV5 specific for E. coli 0157:H7 , LmoM-AG20 (V20) specific for Listeria monocytogenes and EcoP-NAG 24 phage specific for E. coli 045:H2. A transmission electron micrograph for E. coli 045 phase is shown in FIG. 1. Other suitable lytic phages include Sen-AG11 for Salmonella Enteritidis, SnwM-CGG4-1 for Salmonella Newport, CGG 4-1 phage for Salmonella Newport, and FF47 for Mycobacterium avium subspecies paratuberculosis. A transmission electron micrograph for the printed phage Salmonella Newport phage (CGG 4-1 phage) is shown in FIG. 2. Combinations of active phages may be employed as desired. It is understood by one of skill in the art that any lytic phage specific for a desired pathogenic bacteria may be encompassed in various embodiments of the present invention.

Typically, the amount of the active lytic phages for use in the composition for printing is an amount up to about 10¹² PFU/ml . Suitable amounts are from about 10⁵ PFU/ml to about 10¹² PFU/ml, such as about 10⁵ PFU/ml, about 10⁶ PFU/ml, about 10⁷ PFU/ml, about 10⁸ PFU/ml, about 10⁹ PFU/ml, about 10¹¹ PFU/ml, or about 10¹² PFU/ml. Typically, about 10⁹ PFU/ml is used. One of skill in the art may appreciate that lower or higher amounts are envisaged depending on the desired application.

The active lytic phages are provided in a bio-ink composition comprising a surfactant. Suitable surfactants for use may include but are not limited to Triton™ X100, or other similar neutral or non-ionic surfactants of the Triton family, Tween family, and Brij family, typically used in concentrations in the millimolar range, such as for example, 0.5-5 mM.

Useful examples of non-ionic hydrocarbon surfactants include ethers, such as polyoxyethylene nonyl phenyl ether, polyoxyethylene octyl phenyl ether, polyoxyethylene dodecyl phenyl ether, polyoxyethylene alkyl allyl ethers, polyoxyethylene oleyl ether, polyoxyethylene lauryl ether, polyoxyethylene alkyl ethers, polyoxyalkylene alkyl ethers; esters, such as polyoxyethylene oleate, polyoxyethylene distearate, sorbitan laurate, sorbitan monostearate, sorbitan monooleate, sorbitan sesquioleate, polyoxyethylene monooleate, and polyoxyethylene stearate; and glycol surfactants.

Specific examples of non-ionic hydrocarbon surfactants include octylphenoxy polyethoxy ethanols, such as Triton™X-100, X-114, and X-405, available from Union Carbide Co., Danbury, Conn., acetylenic diols such as 2,4,7,9-tetramethyl-5-decyn-4,7-diol and the like, such as Surfynol™GA and Surfynol™CT-136, available from Air Products & Chemicals Co., Allentown, Pa., trimethyl nonylpolyethyleneglycol ethers, such as Tergitol™TMN-10, available from Union Carbide Co., Danbury, Conn., non-ionic esters of ethylene oxide, such as Merpol™SH available from E. I. Du Pont de Nemours & Co., Wilmington, Del., non-ionic esters of ethylene oxide and propylene oxide, such as Merpol™ FH, available from E. I. Du Pont de Nemours & Co., Wilmington, Del., and the like, as well as mixtures thereof.

The active phage bio-ink composition of the invention comprises a viscosity modifier. Examples of suitable viscosity modifiers include but are not limited to aliphatic ketones; stearone; glycerol (acts also as a humectant for stability); 2-hydroxybenzyl alcohol; 4-hydroxybenzyl alcohol; 4-nitrobenzyl alcohol; 4-hydroxy-3-methoxy benzyl alcohol; 3-methoxy-4-nitrobenzyl alcohol; 2-amino-5-chlorobenzyl alcohol; 2-amino-5-methylbenzyl alcohol; 3-amino-2-methylbenzyl alcohol; 3-amino-4-methyl benzyl alcohol; 2(2-(aminomethyl)phenylthio)benzyl alcohol; 2,4,6-trimethylbenzyl alcohol; 2-amino-2-methyl-1,3-propanediol; 2-amino-1-phenyl-1,3-propanediol; 2,2-dimethyl-1-phenyl-1,3-propanediol; 2-bromo-2-nitro-1,3-propanediol; 3-tert-butylamino-1,2-propanediol; 1,1-diphenyl-1,2-propanediol; 1,4-dibromo-2,3-butanediol; 2,3-dibromo-1,4-butanediol; 2,3-dibromo-2-butene-1,4-diol; 1,1,2-triphenyl-1,2-ethanediol; 2-naphthalenemethanol; 2-methoxy-1-naphthalenemethanol; decafluoro benzhydrol; 2-methylbenzhydrol; 1-benzeneethanol; 4,4′-isopropylidene bis(2-(2,6-dibromo phenoxy)ethanol); 2,2′-(1,4-phenylenedioxy)diethanol; 2,2-bis(hydroxymethyl)-2,2′,2″-nitrilotriethanol; di(trimethylolpropane); 2-amino-3-phenyl-1-propanol; tricyclohexylmethanol; tris(hydroxymethyl)aminomethane succinate; 4,4′-trimethylene bis(1-piperidine ethanol); N-methyl glucamine; xylitol; or mixtures thereof. The viscosity modifier is present in the bio-ink composition in any effective amount, so long as not to affect the viability of the phage, such as at least 10% by weight of the bio-ink composition, no more than about 30%, no more than about 15%, or from about 30% to about 55% or from about 35% to about 50%.

Other conventional additives may further be added to the bio-ink composition of the invention so long as it does not affect the viability of the lytic phase contained therein. Other suitable conventional additives may include, for example, dispersants, propellants, biocides, defoamers, slip and leveling agents, plasticizers, antioxidants, humectants, UV absorbers, tackifiers, adhesives, colorants, dyes, conductivity enhancing agents and the like as is understood by one of skill in the art. Combinations of these agents may also be used. Conventional additives may be added in any effective amount as is understood by one of skill in the art, so long as to not affect the viability of the phage contained therein.

Optional polysaccharides may also be added to the active phage bio-ink composition. Any suitable polysaccharide may be used to aid in the stability of the composition is envisaged such as but not limited to trehalose, maltose and the like. The amount may be any amount that does not affect the viability of the phage. Amounts such as for example up to about 10% by weight of the composition, up to about 15% by weight of the composition, up to about 20% by weight of the composition and up to about 50% or more by weight of the composition can be incorporated.

Once the active phage bio-ink composition is made that comprises active lytic phage or combinations of active lytic phage, surfactants, viscosity modifiers and optional conventional additives the active phage bio-ink is printed onto a suitable substrate. In aspects the suitable substrate can be any type of paper. In aspects the paper is a colour-fast paper with printing by using a piezoelectric printer. In one example, ‘ColorLok’ HP paper may be used in conjunction with a Dimatix printer. Printers suitable for use in the present invention are those that do not generate heat. Piezoelectric printers are suitable for use in the present invention. As previously stated, the phages have a net negative charge on their capsids (heads) and positive charge on their tails. Thus cationic papers (substrates) are desired for the oriented immobilization of the lytic phages at the paper surface through heads, leaving the tail fibers free to capture the target bacterium. Printing technology as demonstrated herein now provide for the commercial application of phage-based bioactive paper.

Once the bio-ink composition is printed onto the cationic paper, it is allowed to dry for up to about two hours, and is kept at about 80% relative humidity (rh) or up to about 85% rh to keep the phage active. The paper may be washed in order reduce the number of poorly adsorbed or non-adsorbed phages to reduce background noise. Once made and dried the printed paper can be cut into strips and used as a test strip (dipstick) or other method to bind target bacteria in the sample, after which the strip is transferred to clean broth and incubated. After incubation of about 5 hours the enriched sample can be interrogated via a preferred detection method selected from real-time PCR, isothermal DNA amplification, ELISA, etc. to detect the progeny phage released from the target bacterium. Advantageously, this leads to essentially only detection of the progeny phages and not the immobilized originally added phages.

The printed phage-based bioactive paper typically contains from about 10³ to 10⁹ PFU per cm² of the paper, such as about 10³ PFU/cm², about 10⁴ PFU/cm², about 10⁹ PFU/cm², about 10⁶ PFU/cm², or about 10⁷ PFU/cm². Typically, about 10⁵ PFU/cm² is used.

The paper so produced can be used to produce food-packaging material capable of inhibiting growth of the target bacterium in food during storage. It can be used to package foods, wrap foods, used in the fast food industry, etc. Any type of food can be envisaged. The printed phage-based bioactive paper can be formatted as a test strip for dip testing as described herein, or cut into any shape or size as desired. The active phage bio-ink composition can be printed onto the cationic paper in a variety of patterns on the paper itself for various applications. The cationic material as described herein can be paper, but may also include other cationic paper-based materials and edible films (ie. food casings) used for food packaging.

Several advantages of aspects of the present invention include:
Phages used for printing are inexpensive-manufacturing costs are expected to be low
Scale up of manufacturing is straightforward
Methods of controlled atmosphere packaging to maintain humidity are standard
Easy to use
Detection as low as 10 cfu/g or less
Results within one working day (8 h), using naturally contaminated food
Repeating the same approach but using the more specific STEC phage that was recently isolated and characterized, which is specific against E. coli 045:H2, one of the “Big Six” non-0157 STEC serogroup strains
Printing other phages against various foodborne pathogens (e.g., L. monocytogenes, Salmonella)
Validating the detection system for known pathogen/food combinations: e.g., L. monocytogenes and processed meats; L. monocytogenes and soft cheese; STEC and ground beef, etc.

A limitation to the detection technology is related to the specificity of the phage for the bacteria in question and this is simply controlled by verifying that the phages employed in the invention have the appropriate host range as would be understood by one of skill in the art. When used as packaging material a concern could be possible development of resistance to the phage and the transfer of virulence and/or antimicrobial resistance genes by the phage. These can be overcome by using a cocktail of phages and ensuring that the phage does not contain genes responsible for lysogeny, respectively.

Representative food borne bacterial pathogens for detection and lysis using the present invention may include for example Campylobacterjejuni, Clostridum botulinum, Clostridium perfringens, S. enterica, such as S. Enteritidis and S. Typhimurium, Cronobacter, E. coli such as E. coli 0157:H7 and E. coli 045:H2, Listeria monocytogenes, Vibrio and Shigella.

To summarize, a novel approach has been taken to print active phages on cationic paper to produce a phage-based bioactive paper that can be used to control the growth of foodborne bacteria to enhance food safety. The active phage as used in the composition and printed onto cationic paper is oriented to help ensure that the tail fibers of the phage are free to capture the target bacterium. The oriented phage-based bioactive paper can also be used in various strip formats in methods to detect progeny phage released from target bacterium. Thus the amount of bacteria can be assayed for. The paper so formed is stable over time and can be stored under different conditions such as temperature, low pH and elevated salt concentrations.

Disclosed are materials, compositions, and components that are used for, used in conjunction with, used in preparation for, or are products of the disclosed methods and compositions. These and other materials are disclosed herein, and it is understood that when combinations, subsets, interactions, groups etc. of these materials are disclosed that while specific reference of each various individual and collective combinations and permutation of these compounds may not be explicitly disclosed, each is specifically contemplated and described herein. For example, if a phage is disclosed and discussed and a number of modifications that can be made to a number of molecules including the phage are discussed, each and every combination and permutation of the phage and the modifications that are possible are specifically contemplated unless specifically indicated to the contrary. This concept applies to all aspects of this application including, but not limited to, steps in methods of making and using the disclosed compositions. Thus, if there are a variety of additional steps that can be performed, it is understood that each of these additional steps can be performed with any specific embodiment or combination of embodiments of the disclosed methods, and that each such combination is specifically contemplated and should be considered disclosed.

Unless defined otherwise, all technical and scientific terms used herein have the same meanings as commonly understood by one of skill in the art to which the disclosed method and compositions belong. Publications cited herein and the material for which they are cited are hereby specifically incorporated by reference.

Throughout the description and claims of this specification, the word comprise and variations of the word, such as comprising and comprises, means including but not limited to, and is not intended to exclude, for example, other additives, components, integers or steps.

It is to be understood that the disclosed method and compositions are not limited to specific synthetic methods, specific analytical techniques, or to particular reagents unless otherwise specified, and, as such, may vary. It is also to be understood that the terminology used herein is not intended to be limiting. Note the headings used herein are for organizational purposes only and are not meant to limit the description provided herein or the claims attached hereto.

The following examples are put forth so as to provide those of ordinary skill in the art with a complete disclosure and description of how the phage-based bioactive papers, packing material, compositions, claimed herein are made and evaluated, and are intended to be purely exemplary and are not intended to limit the disclosure.

### EXLAMPLES

### Example 1

### Printing of Bacteriophage on ColorLok Paper for the Detection of E. coli 0157:H7 in Broth and in Spinach using Non-Dipstick Method

### Materials and Methods

Briefly, phage bio-inks were prepared using Triton™ X-100 detergent, CaCl₂ and glycerol. Immobilization was done via Dimatix piezoelectric printer onto ColorLok® paper to detect E. coli. Infectivity trials were done to determine the effect of piezoelectric printing on the phage. All trials and tests were done in triplicates for statistical significance.

### Printing

E. coli 0157:H7 specific lytic phage (rV5) was printed on Color Lok cationic paper by piezoelectric inkjet printing. The bio-ink solution was produced by mixing 30% glycerol, 2 mM Triton X100 and 10⁹ PFU/mL of Rv5. The bio-ink was mixed immediately before printing, injected into the Dimatix printer cartridges, and kept at room temperature for 30 min. The printer was programmed to deposit a drop volume of 10 pl every 20 μm, resulting in 10⁶ PFU of rV5 per cm² of the paper. The printed phage-paper was allowed to dry for 1 hour, cut into 0.5×2.5 cm strips and then stored at 85% relative humidity.

Detection protocols, Non-Dipstick:

A) In Broth Medium

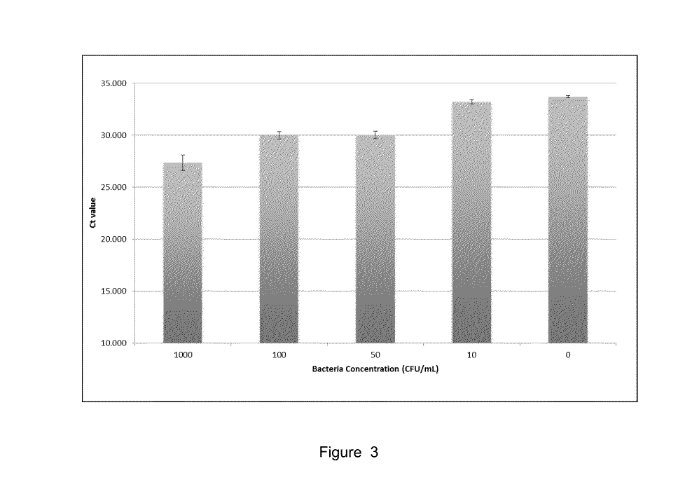

The ability of the phage-paper to detect E. coli 0157:H7 was first tested in inoculated Tryptic Soy Broth (TSB). An overnight culture of E. coli 0157:H7 was serially diluted to 10³, 10², 50, 10 and 0 CFU/mL in TSB. The phage-based bioactive paper was added to each bacterial suspension and allowed to incubate at 37° C. for 20 minutes in Eppendorf tubes. The inoculated broth was then removed and fresh TSB was added to each tube. The samples were then incubated at 37° C. for 5 hours. After the incubation period, the phage-based bioactive paper was removed and 60 uL of chloroform was added. Real-time PCR was performed for each sample under standard conditions, using Power SYBR® Green PCR reagent. The Cycle Threshold (Ct) values for each sample were compared to the control. Detection was considered to be valid if the Ct values were significantly different from the control Ct values (95% Confidence Interval). The experiment was performed in triplicate. Each sample was also serially diluted and 10 uL was spotted on a semi-solid agar overlay of E. coli 0157:H7 to count phages. The phage counts in the samples with bacteria were compared to the control sample (FIG. 3)

B) In Spinach Leaves

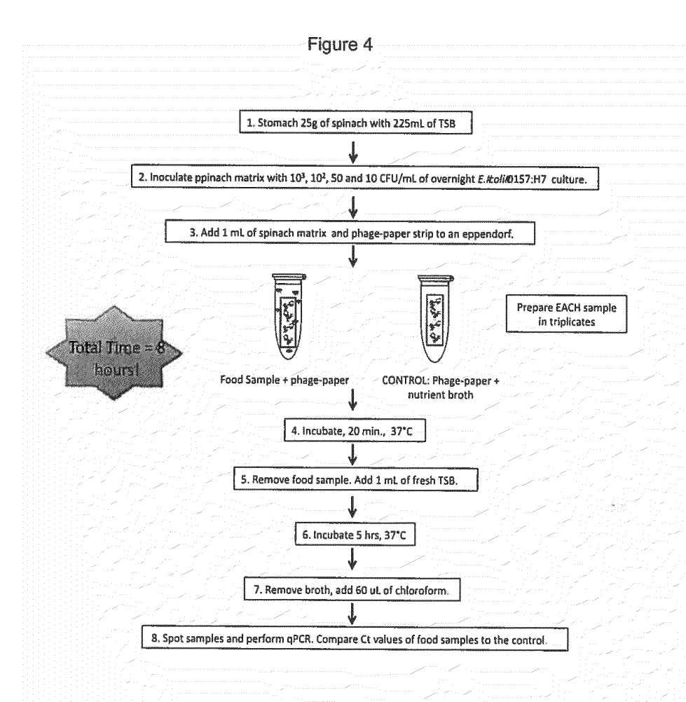

Spinach (25 g) was weighed and added to 225 mL of TSB. The spinach sample was then stomached at 200 rpm for 1 minute and inoculated with E. coli 0157:H7 diluted to 10³, 10², 50, and 10 CFU/mL in TSB. The detection method described above was then followed. (FIG. 4). Again, triplicate experiments were performed.

### Broth Detection Trials

The detection limit for the broth trials was found to be approximately 10 CFU/mL within 8 h, at which concentration the Ct value remained significantly (p<0.05) different from the control Ct value, using a 95% confidence interval (Table 1 and FIG. 3). The number of progeny phage was confirmed by spotting (Table 2), and the numbers of phages released increased with increases in initial concentration of the target organism present.

### Spinach Detection Trials

The detection limit for E. coli 0157:H7 in spinach was found to be approximately 10 CFU/mL within 8 h using the same criteria described above (Table 3). The number of progeny phage was confirmed by spotting (Table 3), and the numbers of phage released increased with increase in initial concentration of the target organism present.

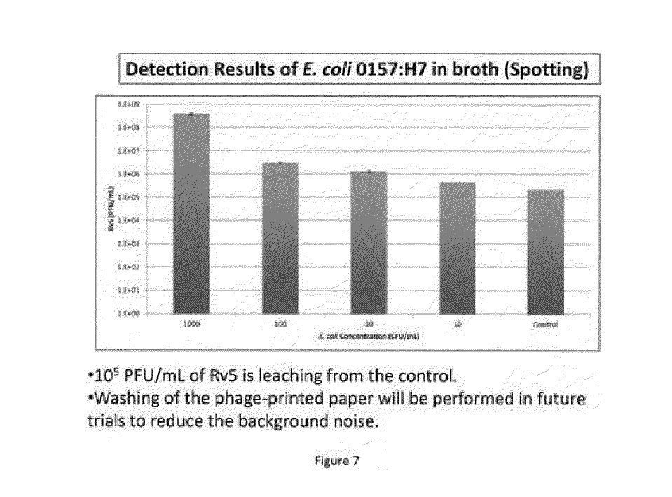

TABLE 1
Detection of phage particles in broth with and without E. coli 0157:H7
using qPCR. (FIG. 8)

| Approximate | | | | |
|---|---|---|---|---|
| Bacteria Count | | Ct Standard | P-value | Number of |
| (CFU/mL) | Ct Mean | Error | (95% CI) | replicates |
| 1000 | 27.35 | 0.73 | 2.2E−08 | 9 |
| 100 | 29.99 | 0.35 | 3.35E−09 | 9 |
| 50 | 30.80 | 0.37 | 5.4E−07 | 9 |
| 10 | 33.24 | 0.22 | 0.03 | 9 |
| Control | 33.71 | 0.09 | | 9 |

TABLE 2
Detection of phage particles in broth with and without E. coli 0157:H7
by spot test technique. (FIG. 7)

| Approximate Bacteria | | Ct Standard | Number |
|---|---|---|---|
| Count (CFU/mL) | Mean RvS Titre | Error | of replicates |
| 1000 | 6.23E+06 | 1.86E+06 | 3 |
| 100 | 5.37E+05 | 2.82E+05 | 3 |
| 50 | 9.48E+04 | 4.84E+04 | 3 |
| 10 | 2.27E+04 | 1.86E+04 | 3 |
| Control | 3.80E+03 | 6.11E+02 | 3 |

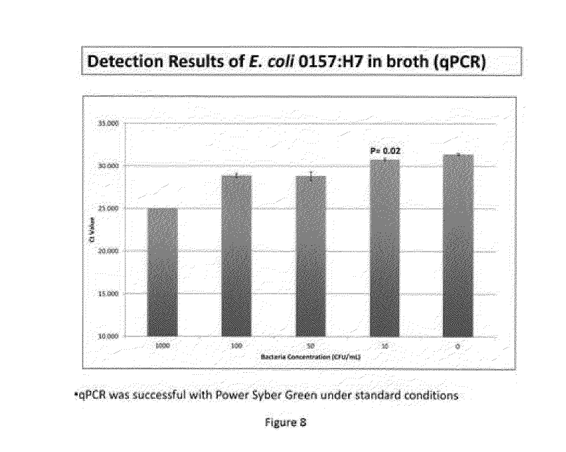

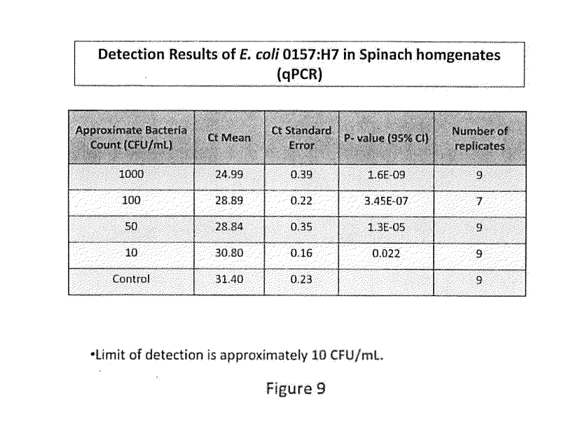

TABLE 3
Detection of phage particles in spinach leaves homogenate with
and without E. coli 0157:H7 by qPCR. (FIG. 9)

| Approximate Bacteria | | Ct Standard | P-value | Number of |
|---|---|---|---|---|
| Count (CFU/mL) | Ct Mean | Error | (95% CI) | replicates |
| 1000 | 26.45 | 0.40 | 5.55E−7 | 9 |
| 100 | 28.39 | 0.13 | 2.63E−6 | 9 |
| 50 | 29.76 | 0.19 | 0.02 | 9 |
| 10 | 29.676 | 0.14 | 0.007 | 9 |
| Control | 30.451 | 0.23 | | 9 |

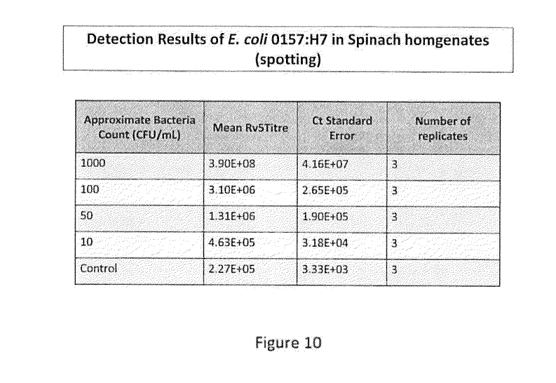

TABLE 4
Detection of phage particles in spinach leaves homogenate
with and without E. coli 0157:H7 by spot test technique. (FIG. 10)

| Approximate Bacteria | Mean RvS | Ct Standard | Number of |
|---|---|---|---|
| Count (CFU/mL) | Titre | Error | replicates |
| 1000 | 1.07E+07 | 3.53E+05 | 3 |
| 100 | 8.87E+05 | 5.28E+05 | 3 |
| 50 | 3.23E+05 | 1.21E+05 | 3 |
| 10 | 1.73E+05 | 3.28E+04 | 3 |
| Control | 1.45E+05 | 4.65E+05 | 3 |

### Example 2

### Printing of Bacteriophage on ColorLok Paper for the Detection of E. coli 0157:H7 in Broth and in Spinach Homogenates using Dipstick Method

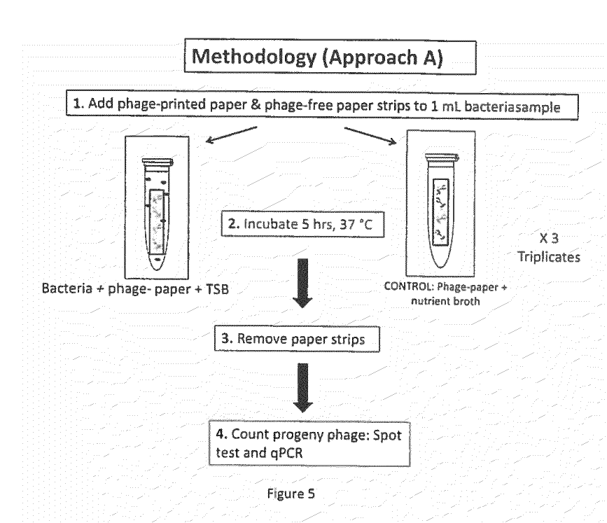

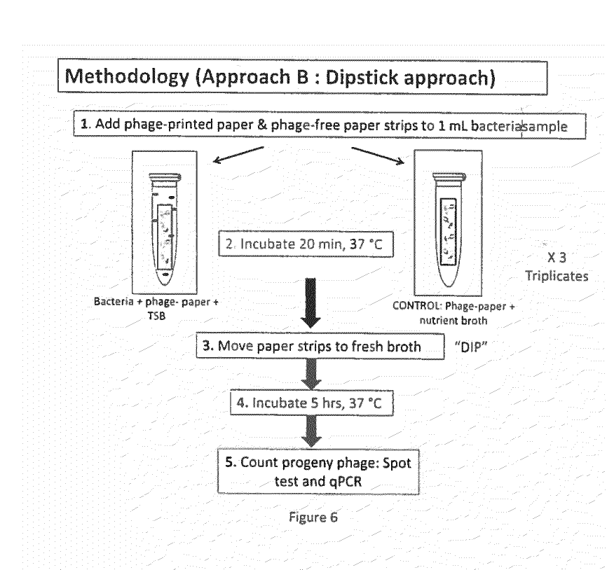

Materials and Methods—similar to that of example 1. Two methodologies were used. Approach A (FIG. 5, spotting technique) and Approach B (FIG. 6, dipstick approach). The bio-ink containing 10⁹Pfu.m1 phage was combined with 2mM Triton X100 and 30% glycerol. The bio-ink mixture was injected into a cartridge and rested inverted for about 30 minutes. The printer was set to 40V, drop space 20 μm, meniscus vacuum 11.43 HO. The ColorLok paper was taped to the surface and the paper printed. The printed phage-based bioactive paper was stored at 80% RH in petri dishes. the paper was cut into 2.5 cm×0.5 cm strips just before use.

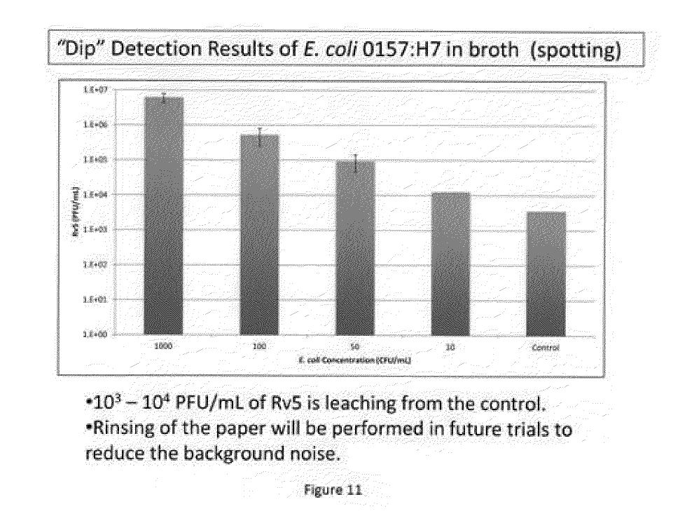

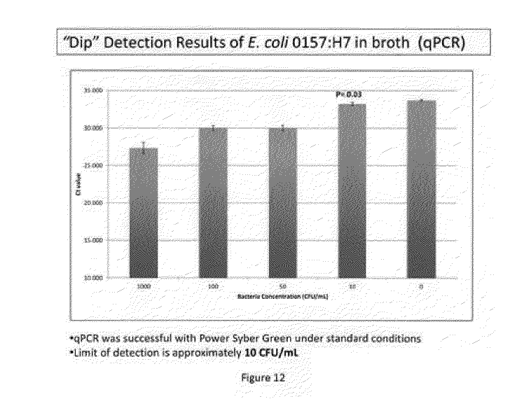

Using the spotting technique, E. coli0157:H7 was detected in broth using spotting (FIG. 7) and qPCR (FIG. 8). The detection results of E. coli 0157:H7 in spinach homogenates using qPCR is shown in FIG. 9 and using spotting is shown in FIG. 10. The dip detection results of E. coli 0157:H7 in broth using spotting is shown in FIG. 11 and qPCR in FIG. 12. The bacterial counts are shown in Table 5.

TABLE 5
Detection Results of E. coli 0157:H7 in broth

| Approx- | | | | | | |
|---|---|---|---|---|---|---|
| imate Bac- | Rv5 | log | | | | #of |
| teria Count | Titre Log | (pfu/mL) | Ct | Ct | | repli- |
| (CFU/mL) | (PFU/mL) | SE | Mean | SE | P- value | cates |
| 1000 | 6.79 | 0.32 | 27.35 | 0.73 | 2.2E−08 | 9 |
| 100 | 5.62 | 0.21 | 29.99 | 0.35 | 3.35E−09 | 9 |
| 50 | 4.64 | 0.50 | 30.80 | 0.37 | 5.4E−07 | 9 |
| 10 | 3.99 | 0.40 | 33.24 | 0.22 | 0.03 | 9 |
| Control | 3.57 | 0.06 | 33.71 | 0.09 | | 9 |

Limit of detection is approximately 10 CFU/mL.

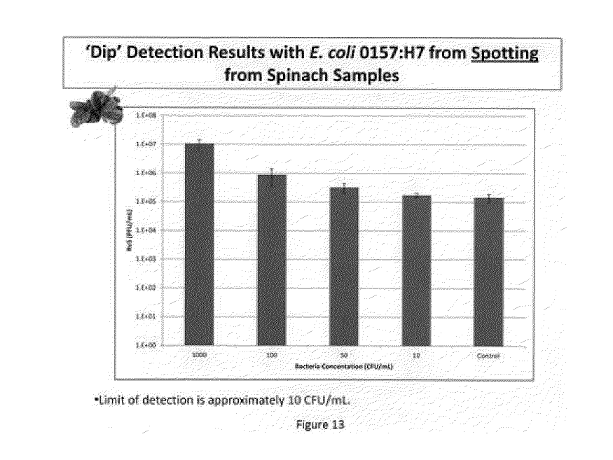

The dip detection results with E. coli 0157:H7 from spotting from spinach samples is shown in FIG. 13 and using qPCR in FIG. 14. The bacterial counts of E. coli 0157:H7 in spinach homogenates is shown in Tables 6 and 7.

TABLE 6
Detection Results of E. coli 0157:H7 in Spanish homogenates (Trial 1)

[symbol]

| Approx- | | | | | | |
|---|---|---|---|---|---|---|
| imate Bac- | Rv5 | log | | | | # of |
| teria Count | Titre Log | (pfu/mL) | Ct | Ct | | repli- |
| (CFU/mL) | (PFU/mL) | SE | Mean | SE | P- value | cates |
| 1000 | 6.96 | 0.18 | 26.45 | 0.40 | 5.55E−7 | 9 |
| 100 | 5.73 | 0.34 | 28.39 | 0.13 | 2.63E−6 | 9 |
| 50 | 5.43 | 0.19 | 29.76 | 0.19 | 0.02 | 9 |
| 10 | 5.22 | 0.09 | 29.676 | 0.14 | 0.007 | 9 |
| Control | 5.09 | 0.28 | 30.451 | 0.23 | | 9 |

Limit of detection is approximately 10 CFU/mL.

TABLE 7
Detection Results of E. coli 0157:H7 in Spinach homegenates
(Trial 2 June 26th)

[symbol]

| Approx- | | | | | | |
|---|---|---|---|---|---|---|
| imate Bac- | Rv5 | log | | | | # of |
| teria Count | Titre Log | (pfu/mL) | Ct | Ct | | repli- |
| (CFU/mL) | (PFU/mL) | SE | Mean | SE | P- value | cates |
| 1000 | 7.30 | 0.14 | 23.97 | 1.23 | 7.67E−4 | 9 |
| 100 | 6.18 | 0.15 | 26.44 | 0.94 | 0.006 | 9 |
| 50 | 6.18 | 0.02 | 26.46 | 0.91 | 0.006 | 9 |
| 10 | 5.38 | 0.11 | 27.90 | 0.72 | 0.03 | 9 |
| 1 | 4.90 | 0.12 | 30.55 | 1.19 | — | 9 |
| Control | 5.30 | 0.13 | 30.22 | 0.98 | — | 9 |

Limit of detection is approximately 10 CFU/mL.

Storage of the phage-based bioactive paper did not negatively affect the ability to detect E. coli 0157:H7 since bacteria were still detected after one week of storage at 80-85% relative humidity as shown in Table 8.

TABLE 8
Detection Results with E. coli 0157:H7 after
1 week storage (80-85% Relative Humidity)

| Approx- | | | | | | |
|---|---|---|---|---|---|---|
| imate Bac- | Rv5 | log | | | | # of |
| teria Count | Titre Log | (pfu/mL) | Ct | Ct | | repli- |
| (CFU/mL) | (PFU/mL) | SE | Mean | SE | P- value | cates |
| 1000 | 2.71 | 0.49 | 30.04 | 0.41 | 0.0003 | 9 |
| 100 | 2.52 | 0.12 | 31.12 | 0.34 | 0.01 | 9 |
| 50 | 2.40 | 0.17 | 32.07 | 0.61 | 0.29 | 9 |
| 10 | 2.04 | 0.04 | 32.27 | 0.33 | 0.35 | 9 |
| Control | 2.46 | 0.19 | 30.22 | 0.98 | — | 9 |

Limit of detection is approximately 10³ CFU/mL.
Detection was not possible after 2 weeks

### Example 3

### Effect of Bioink Composition on Listeria Phage LmoM-AG-20

### Materials and Methods

Phage bio-inks were prepared using Triton™ X-100 detergent, CaCl₂ and glycerol. Immobilization was done via Dimatix piezoelectric printer onto ColorLok® paper to detect L. mono (Table 9). Dounting of the phage was done to determine the effect of the bio-ink composition on the phage. All trials and tests were done in triplicates for statistical significance.

TABLE 9
Detection and capture experimental procedure

| | Detection | | Capture |
|---|---|---|---|
| 1. | Incubate O/N Culture | 1. | Incubate O/N Culture |
| 2. | Add 1 mL to Eppendorf tubes | 2. | Add 1 mL to Eppendorf tubes |
| 3. | Add phage and control paper | 3. | Add phage and control paper |
| 4. | Detection after 5 hours | 4. | After 20 minutes remove broth |
| | incubation at 37° C. | | and move phage paper to fresh |
| 5. | Remove broth | | broth (Dip) |
| 6. | Spot progeny phage, perform | 5. | Detection after 5 hrs. incubation |
| | RT-PCR | 6. | Remove broth |
| | | 7. | Spot progeny phage, perform |
| | | | RT-PCR |

TABLE 10
Infectivity/Biocontrol experimental procedure

| | | |
|---|---|---|
| Biocontrol/ | | 10³ CFU/ml L. mono + Phage Paper |
| Infectivity | | 10³ CFU/ml L. mono + Control Paper |
| in triplicates | | 10³ CFU/ml L. mono + 10⁶ |
| (24 hours) | {close oversize brace} | PFU/ml V20 Phage |
| | | Phage Paper + TSB Broth |
| | | 10³ CFU/ml L. mono |

Listeriophage V20 is highly infective for Listeria monocytogenes and used in this trial to be tested as a novel detection method. The trials done indicated that V20 Listeriophage can be suspended in the bio-ink solution, remain infective after printing and remain infective after 2 weeks storage at 4° C. (Table 11). The trials confirmed the printed Listeriophage V20 has utility for packaging foods such as ready to eat meats and cheeses susceptible to Listeria.

TABLE 11
Titers in PFU/ml of V20 Phage and Bioinks used in Printing Trials

| | | Bioink | Pure | Crude | Pure | Crude |
|---|---|---|---|---|---|---|
| Pure | Crude | after 2 | Phage | Phage | Phage | Phage |
| Phage | Phage | weeks | Bioink | Bioink | Leached | Leached |
| 3.1 × 10¹⁰ | 2.5 × 10⁹ | 8.0 × 10⁸ | 1.5 × 10⁹ | 8.0 × 10⁸ | 4 | 1 |

### Example 4

### Infectivity of Immobilized Phaqe for E. coli O45 and Salmonella Newport

Materials and methods were similar to that of the previous examples. Phage bio-inks were prepared using Triton™ X-100 detergent, CaCl₂ and glycerol. Immobilization was done via Dimatix piezoelectric printer onto ColorLok® paper to detect E. coli O45 (phage shown in FIG. 1). As seen in FIG. 15, the printed phage caused inhibition to the bacterial growth to below the detection limit of 1 log CFU/ml when incubated with its host for 18 hours at 37° C.

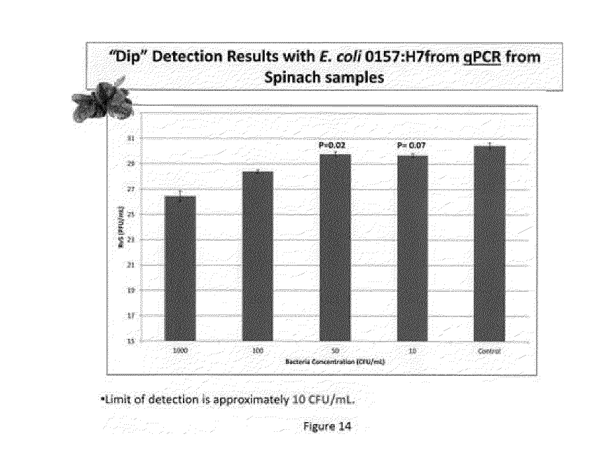

The experiment was repeated using Salmonella Newport phage (CGG 4-1 phage, seen in FIG. 2). The results shown in FIG. 16 indicate that the printed phage caused around 4.5 and 4 log₁₀ reduction in the bacterial count after one day and one week storage, respectively after 18 hours incubation with its host.

### Example 5

### Immobilization of Bacteriophages on Paper to Make Bioactive Food Packaging for Control of Food-Borne Pathogens

On the basis that phages may have a net negative charge on their capsids and positive charge on their tails, they have herein been developed for oriented immobilization of the phages at the paper surface.

Printing and coating of phages onto paper has been now developed for phage-based packaging material. Gravure printing, blade coating, thermal and piezoelectric inkjet printing are the technologies used for immobilization of phages of different morphologies from the Leviviridae, Siphoviridae and Myoviridae families. Different ink compositions and paper types were used for immobilizing the bacteriophages onto the paper.

### Materials and Methods

### Bacteriophages and Bacteria

All phages and bacteria were obtained from the Canadian Research Institute for Food Safety (CRIFS) culture collection (Table 12). All bacterial host cultures were stored in 25% glycerol at −80° C. Working cultures were streaked onto Tryptic Soy Agar (TSA, BD Diagnostics, Mississauga, ON) and Liquid cultures were prepared by subculturing into Tryptic Soy Broth (TSB, BD Diagnostics). The bacterial cultures were sub-cultured from −80π C. stock cultures on 2 monthly basis.

TABLE 12
Bacteriophages used for printing and
coating, and their respective hosts.

| | | | | Host CRIFS |
|---|---|---|---|---|
| | Abbrevi- | | | Culture |
| Phage | ation | Family | Host | Collection # |
| StyM-AG6 | AG6 | Myoviridae | S. | C1077 |
| | | | Typhimurium, | |
| | | | DTI04 | |
| SenS-AG11 | AG11 | Siphoviridae | S. Enteritidis | C417 |
| LmoM-AG20 | AG20 | Myoviridae | L. | C1301 |
| | | | monocytogenes | |
| | | | P7, | |
| EcoM-AGI0 | AG10 | Myoviridae | E. coli | C899 |
| | | | 0157:H7, | |
| | | | ATCC 43888 | |
| T4 | — | Myoviridae | E. coli B | ATCC |
| | | | | 11303 |
| MS2 | — | Leviviridae | E. coli Famp | — |

### Bacteriophage Propagation

A modification of the protocol described by Sambrook and Russell (2001) was used for phage propagation. Phages were added to overnight cultures (16-18 h incubation time) of their respective hosts at a multiplicity of infection (MOI) of 1-10. Phages were allowed to adsorb to the host cells for approximately 15 min. Molten semisolid TSA (0.5% agar) tempered to approximately 50-55° C. and 5 ml was added per plate. This mixture was added to the surface of TSA plates to form a double-layer agar plate. Plates were allowed to solidify then incubated upright at 25° C. for 16-24 h. Five to ten plates were used for propagation. Phage buffer (0.74 g CaCl, 2.5 g MgSO47H2O, 0.05 g gelatin, enzymatic, 1M Tris.HCl, pH7.5 (6 ml) in a final volume of 11double deionised water) was added to plates showing confluent lysis at 4 ml per plate. The top agar and buffer mixture was scraped off into a sterile tube and another 1 ml phage buffer was added to each plate to wash off any remaining semisolid TSA and the resulting suspension collected into the sterile tube. The tube was placed on ice for at least 15 min. This semisolid agar layer/buffer mixture was centrifuged at 7,000 g for 20 min to precipitate the agar and bacterial cells. The supernatant was removed and filter sterilised through a 0.45 μm pore size, syringe filter (Fisher Scientific). Phage lysates were titred using the overlay method described below then stored at 4° C.

For large scale propagation, one litre of sterile TSB was inoculated with 5 ml fresh overnight culture of host bacteria and incubated for approximately 2 h at 37° C. (0D600 about 0.2, which is approximately 10⁸ cfu/ml). Bacteriophage stock lysate was added at 1 ml/l and incubated overnight at 25° C. then centrifuged at 7,000 g for 20 min. The supernatant was then filter-sterilised with a Corning 0.45 μm sterile bottle top filter unit (Fisher Scientific). Phage lysates were titred using the overlay technique according to Sambrook and Russell (2001) then stored at 4° C.

### Overlay Method to Determine Bacteriphage Titre

Host bacteria were incubated overnight at 37° C. in TSB. One hundred microlitres of host bacteria were added to 5 ml semisolid TSA then carefully pipetted onto a TSA plate and left for 15 min to solidify. Ten-fold dilutions of phage lysate were made and 10 μl were spotted on the surface of the bacterial lawn. Plates were incubated overnight in an upright position at 25° C. The number of plaques was counted after incubation and the titre determined by the following equation:

$$Phage Titre = \frac{Average \# plaques}{dilution plated \times volume plated}$$

### Gravure Printing and Blade Coating of Phages

Morphologically different phages (AG 11, AG20, MS2 and T4), were immobilized on paper by gravure printing and blade coating using equipment at L'Université du Québec à Trois-Rivières. Following application of the phages, the paper was dried, cut and then stored at approximately 80-85% relative humidity (RH) in an air-tight container.

### Gravure Printing

Gravure printing was performed with an IGT Global Standard Tester 2 printability unit (IGT Testing Systems, Arlington Heights, Ill., at Innofibre, Cëgep of Trois-Rivières (QC). Gravure printing experiments were performed on lightweight coated (LWC) commercial paper provided by Centre International de Couchage (CIC), Trois-Rivières (QC) with a basis weight or grammage of 35 g/m2 with a clay and calcium carbonate coating according to the protocol used for printing T4 (Jabrane et al., 2009). A bio-ink solution was formulated containing a 1:1 ratio of phage and 4% carboxy-methylcellulose sodium salt, medium viscosity (Sigma-Aldrich,Canada Co., Oakville, ON) to give a viscosity of 50 mPa·s. Phages AG11, AG20, MS2 and T4 were used for printing. An IGT gravure disc from IGT Testing Systems numbered 402.151.412 was used. It was engraved with 40 lines/cm, screen angle of 53° and contained 4 engravings at cell depths 65 μm, 70 μm, 75 μm and, 80 μm, corresponding to cell volumes of 14 ml/m2, 17 ml/m2, 20 ml/m2, and 25 ml/m2, respectively. Printing was conducted at a velocity of 0.2 m/s and printing pressure equivalent to applied pneumatic force of 600 N onto paper (50×300 mm).

### Blade Coating

Blade coating was performed with a Cylindrical Laboratory Coater, CLC-7000 (Simutech Group, Toronto, ON), at Innofibre. This instrument is designed to reproduce the complete coating process on a laboratory scale at speeds (up to 800 m/min) comparable to pilot machines. Blade coating was done on a softwood mechanical pulp base paper of 47g/m² basis weight. The bioactive basis weight after coating was 1 g/m². The bioactive coating solution was formulated with the phages suspended in 5% pork type A gelatin (Fisher Scientific) and 3% CMC solution (viscosity of 100 mPa·s at 45° C.) prior to coating at a speed of 400 m/min. CLC-7000 built-in high intensity 36 kW infrared lamps were used to dry the phage-coated papers for 30 sec. The temperature of the phage-coated paper after drying was 66° C. as measured by an OMEGASCOPE OS532 infrared thermometer with laser sight (OMEGA Engineering Inc., Laval, QC).

### Inkjet Printing of Bacteriophages

Bacteriophages AG6, AG10, AG11, and AG20 were printed onto uncoated Whatman No.1 paper and hydrogel and polyvinylamine (PVAm)-coated cationic paper and commercially available cationic ColorLok paper by thermal and piezoelectric inkjet printing. Paper samples were then left to dry on a clean bench, and then stored in an air tight container at 80-86% RH and room temperature for one day prior to analysis. Printing experiments were conducted using equipment at McMaster University, Hamilton, ON.

### Cationic Paper Coating for Inkjet Printing

Two cationic coated papers were prepared at McMaster University using Hydrogel (0.5%) and 0.5% PVAm (MW, 45,000, BASF, Mississauga, ON). The PVAm solution was prepared and Whatman No. 1 paper was immersion coated by soaking the paper for 10 min and then washed by dipping into distilled water. The coated paper was gently placed on blotting paper and left to dry in air overnight. Hydrogel-coated paper was prepared using two hydrogel solutions containing 20% by wt poly oligoethylene glycol methacrylate-co-adipic acid dihydrazide-co-dimethylamino ethyl acrylate (OEGMA-co-ADH-co-DMAEA) and 20% by wt poly(OEGMA-co-aldehyde). Both solutions were diluted to 1% in PBS prior to use for paper coating. Whatman paper was immersed in each of the 2 solutions for 10 min and dried for about 15 min in air on blotting paper between the immersion applications.

### Thermal Inkjet Printing

Inkjet printing was done onto Whatman No. 1 paper sheets. A Canon Pixma MP280 (Canon Canada Inc., Toronto, ON) thermal inkjet printer, with commercially available ink cartridges (Canon PG-210 ink cartridge, Black) was used. The bio-ink was formulated using 10⁹ pfu/ml of the phages and Triton X100, 2 mM (Fisher Scientific). The cartridge was opened and the seal between the small main ink compartment and the ink reservoir was broken. The ink cartridge was emptied and rinsed thoroughly with water. The clean cartridge was dried with air. About 0.8 ml of phage bio-ink were added to the cartridge using a pipette before installing it in the printer.

The printer was set to print in grayscale with high quality. For double layer printing the cartridge was topped with additional ink between the printing of the first and second layers to ensure the ink would not run out while printing. Approximately 236 drops/cm were printed for high quality prints at about 25 pl per drop.

### Piezoelectric Inkjet Printing

Inkjet printing was done onto Whatman No. 1 paper sheets, Whatman No. 1 paper coated with 0.5% PVAm, Whatman No. 1 coated with 20 wt % poly(OEGMA-co-ADH-co-DMAEA) in 1% PBS and 20 wt % poly(OEGMA-co-aldehyde), and HP FSC-Certified Multipurpose Paper with Colorlok technology (Hewlett-Packard, Mississauga, ON). Printing was conducted using a Dimatix Materials Printer DMP 2800 (Fujifilm Dimatix Inc., Santa Clara, CA, USA). The bio-ink contained the same phages that were used for thermal inkjet printing (10⁹ pfu/ml) suspended in 30% glycerol and 2 mM Triton X-100. Approximately 500 drops/cm were printed at about 10 pl per drop with printing parameters—firing voltage: 40V; firing frequency: 5 kHz; drop space: 20 μm; meniscus vacuum: 11.43 cm H2O(in Water Column). All jets were set to fire. Nozzle cleaning cycles were performed approximately every 120 sec. Bio-inks formulated for piezoelectric inkjet printing were printed for approximately 15 min directly into a Petri dish for each phage. The titres of the bio-inks before printing and that collected after printing were compared to determine any possible shearing effects during printing.

The bio-inks used for piezoelectric inkjet printing were also pipetted onto ColorLok paper circles (2.5 cm diameter) at 100 μl per paper circle as a control to determine potential shearing effects by piezo printing. This volume provided approximately four times the amount delivered by piezoelectric inkjet printing. The paper was left to dry for 1 to 2 h in a horizontal laminar flow clean bench and then stored at 80-85% RH.

Determination of Infectivity of Bioactive Paper and Phage Diffusion from Paper

The infectivity of the phage-based paper was determined by the overlay technique and/or in liquid media. The bioactive paper was cut into circles (2.5 cm diameter) and transferred using sterile forceps to the surface of semisolid agar inoculated with the relevant bacterium. The medium was incubated in an upright position overnight at 25° C. for E. coli B, L. monocytogenes and Salmonella and 37° C. for E. coli famp. Plates were visually examined for zones of growth inhibition around the periphery of the paper containing phage after the incubation period. The diameters of the zones of inhibition were measured using Image J software (http://rsb.info.nih.gov/ij/).

Infectivity of the phage incorporated in the paper was determined quantitatively using the following method. The host bacterium was incubated for 18 h at 37° C. and diluted to achieve a final count of 10³ cfu/ml in Phosphate Buffered Saline (PBS), (Fisher Scientific). A paper disk (2.5 cm diameter) containing the phage was added to 9 ml of the bacterial suspension and incubated for 18-24 h without shaking. The count of the target bacterium in the broth was determined by plating ten-fold serial dilutions in phosphate buffered saline onto MacConkey Agar (BD Diagnostics, Mississauga, ON), Oxford Agar (Oxoid, Nepean, ON) and XLD (BD Diagnostics) for enumerating E. coli, L. monocytogenes and Salmonella , respectively. Modified Brilliant Green Agar (Oxoid) was also used for enumerating surviving Salmonella in the presence of inkjet printed phage-based paper. The appropriate medium was inoculated with 100 μl of the appropriate dilution of the culture containing phage-based paper, and spread across the agar surface using a sterile spreader. Plates were retained in an upright position until the inoculum was absorbed by the agar then they were inverted and incubated for 18-24 h at 37° C. Positive controls were prepared using paper without phages. After the incubation period, the plates were examined for typical colonies of each host bacterium on the selective plates. Bioactive paper samples were also stored in 5 ml phage buffer for approximately 24-48 h for gravure printed and blade coated papers or for six hours for inkjet printed papers prior to investigating the ability of phages to diffuse from the paper. Diffusion of phages from the bioactive paper was determined by adding the bioactive paper to 5 ml of sterile distilled water and the phage titre was determined by the overlay technique as previously described.

### Stability of Bioactive Paper

The printed or coated phage-based paper was stored at room temperature (approximately 21° C.-25° C.) in an air-tight container. Saturated barium chloride was used to create a relative humidity (RH) of 80-86%. Additionally, samples processed by inkjet printing were also stored in Petri dishes at room temperature under normal atmospheric conditions (50% RH approximately). Phage infectivity was determined by the overlay technique and/or in broth with respective host bacteria after 1 month for inkjet printed paper or monthly up to 2 months for gravure printed paper and blade coated paper according to the procedure described in the previous section. The stability of ColorLok phage-based paper printed by piezoelectric inkjet printing was determined from 2 separate printing experiments. The estimated number of infective phages deposited on the paper by printing or pipetting was determined by theoretical and experimental estimates outlined below.

### Theoretical and Experimental Estimates of Number of Phages Particles on the Printed and Pipetted Bioactive Paper

The number of infective phage particles deposited on the paper was determined theoretically and experimentally. The approximate amount of bio-ink delivered by printing and the initial phage titres were known, therefore estimated phage amounts on the paper were calculated. Experimental estimates were conducted by investigating the effect of different free phage concentrations on the host bacteria then comparing the inhibitory effect with that obtained for the phage-based paper. Additionally, phage AG11, which is the only sequenced phage used in this study, was printed onto ColorLok paper then the DNA was extracted and quantified by qPCR. The number of infective phages on the 2.5 cm diameter phage-based paper was determined by investigating the effect of 6 different free phage concentrations for each phage on 10³ cfu/ml of the respective host bacterium. Ten-fold dilutions of free phages in phage buffer (10³-10⁸ pfu/ml) were each added to 10³ cfu/ml of the respective host bacterium in TSB then incubated at 25° C. for 18-24 h. The surviving bacteria were counted after plating ten-fold serial dilutions of the broth onto selective media as outlined above. The host bacterial counts from the free phage and immobilized phage experiments were compared to estimate the number of infective phage particles present on the paper.

The experimental estimate of the amount of AG11 printed onto ColorLok paper was determined by extraction of the phage DNA from the paper. The amount of DNA present on the paper was determined by the Nanodrop 1000 spectrophotometer (Thermo Scientific, Wilmington, Denver) and qPCR. DNA of AG11 phages printed on the paper (2.5 cm diameter) were extracted using Plant/Fungi DNA Isolation Kit according to manufacturer's instructions (Norgen, Biotek Corp., Thorold, ON). Briefly, phage AG11 was treated with lysis and binding solution while on the paper then the lysate was collected on the spin column, washed, eluted, collected and stored at 4° C. Quantitative real-time PCR (qPCR) was used to detect and estimate the amount of DNA present on the paper samples. Six replicates were conducted. Primers flanking a unique sequence in AG11 were designed using Primer 3 plus Software (Bioinformatics) and synthesised by Laboratory Services, University of Guelph. The primer sequences were:

Forward Primer: AAAGCGCATCAGAACATGAA
Reverse Primer: GCGCAGGTATTCGAGTTGA

The reaction mixture (20 μl total volume) contained 10 μl SYBR Select Master Mix (Applied Biosystems, Life Technologies, Burlington, ON) and 0.5 μM of each primer (4 μl) and 6 pl of extracted AG11 template. The qPCR conditions consisted of 95° C. for 15 sec and 60° C. for 1 min for 40 cycles with a final melt cycle and the qPCR was performed using a ViiA7 (Life Technologies).

### Statistical Analysis

The experiments were conducted as three independent studies and each analysis was performed in triplicate. Log₁₀ transformation of the bacterial counts was performed for statistical analysis, which was conducted by univariate ANOVA tests using IBM SPSS statistics, version 21 software. Differences between means were considered as significant at P<0.05.

### Results:Gravure Printing and Blade Coating of Phages

The infectivity of the phages in the developed inks and on the printed and coated paper was determined. The titres of the phages before and after resuspension in the inks were compared. The results showed that there was no adverse effect of the inks used in the coating and printing on the phage activity (Table 13).

TABLE 13
Comparison of phage titres before and after suspension in bio-inks.
Blade coating bio-inks were composed of phage, gelatin and CMC while
gravure printing bio-inks were composed of phage and CMC.

| | | Phage titre in | Phage titre in |
|---|---|---|---|
| | Original phage | Blade Coating Ink | Gravure Printing |
| Phage | titre (pfu/ml) | (pfu/ml) | Ink (pfu/ml) |
| MS2 | 5.0 × 10⁹ | 3.0 × 10⁸ | 8.0 × 10⁸ |
| AG11 | 1.0 × 10¹¹ | 4.0 × 10⁹ | 5.0 × 10¹¹ |
| AG20 | 5.0 × 10⁸ | 5.5 × 10⁷ | 2.0 × 10⁷ |
| T4 | 1.0 × 10¹⁰ | 4.0 × 10⁹ | 1.3 × 10¹⁰ |

Determination of Infectivity of Bioactive Paper and Phage Diffusion from Paper

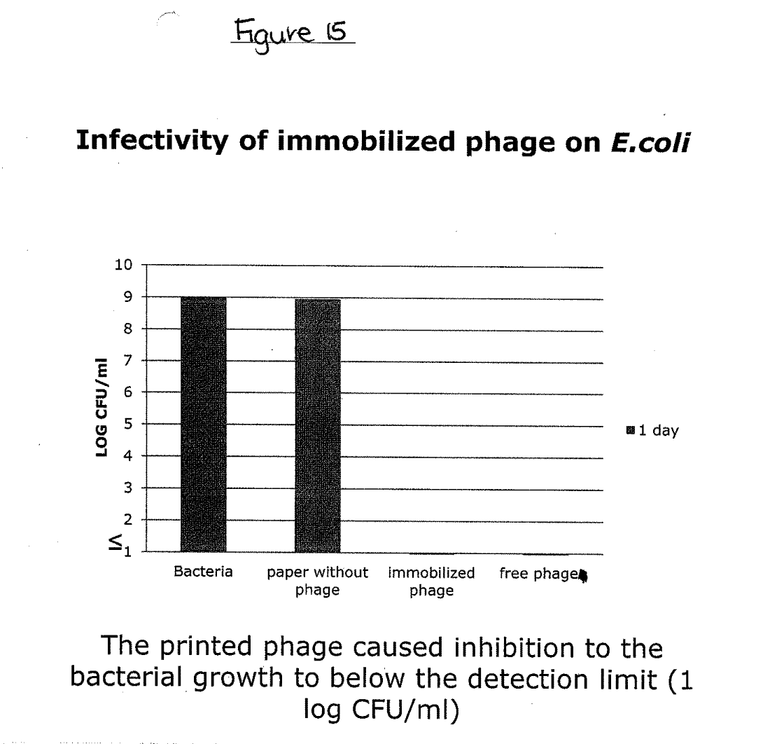

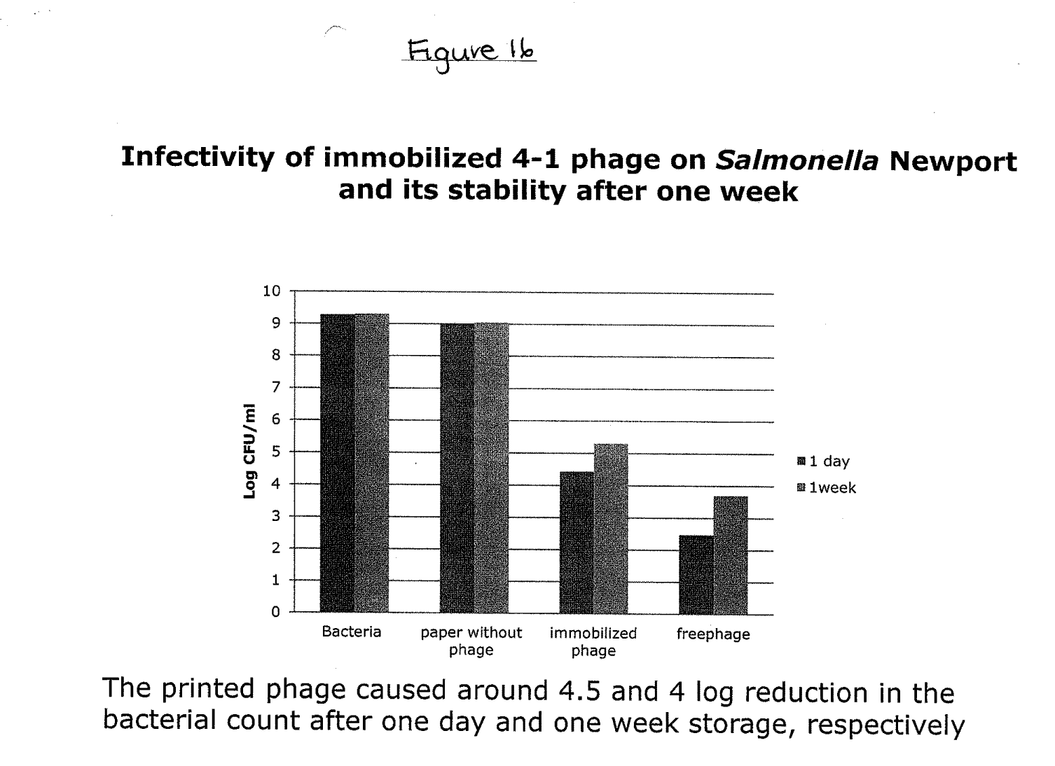

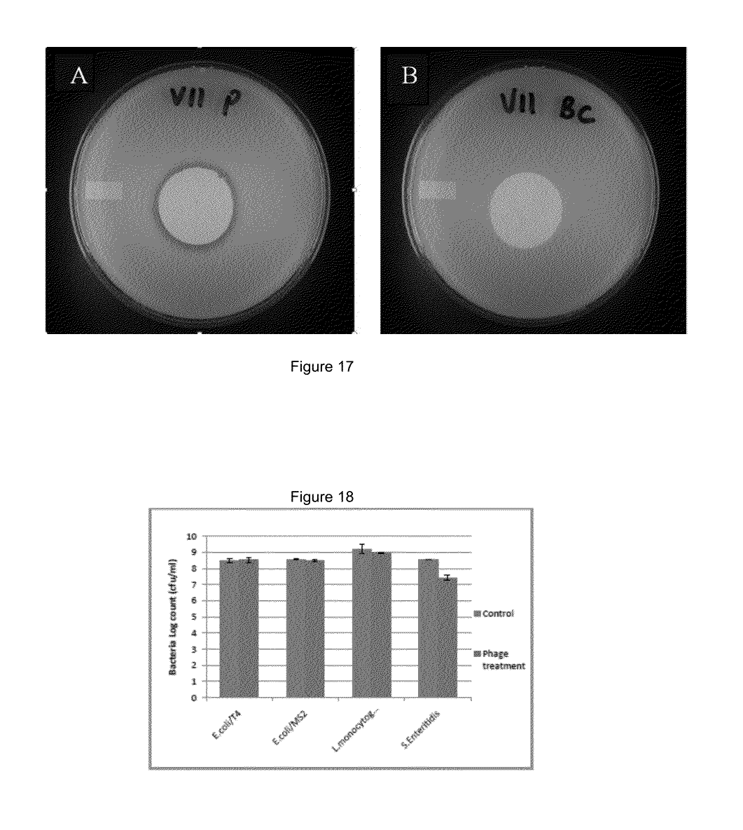

Infectivity of printed and coated phage-based paper was determined by observation of inhibition zones by the overlay method and quantitative assessment of the growth of host bacteria in broth. Gravure printed phage AG11 was the only phage that demonstrated infectivity by the overlay method after blade coating and gravure printing (Table 14, FIG. 17). The AG11 phage bioactive papers reduced counts of S. Enteritidis by 1.16 log₁₀ cfu/ml for the gravure printed papers and by 0.76 log₁₀ cfu/ml for papers produced by blade coating (FIG. 18). Bacterial growth was observed on paper surfaces and in broth cultures without host bacteria (negative controls), indicating contamination of paper during and/or subsequent to printing and coating.

TABLE 14
Infectivity of printed and coated phage bioactive paper. Gravure and
blade coated bioactive paper was placed on lawn of host bacterium
and incubated for 18-24 h at 25° C. The diameter of clear zones
of lysis around the bioactive paper were measured. The activity was
assessed after initial printing and coating then monthly for 2 months
after storage at room temperature at approximately 80% RH.

| Phage in | | | | | | |
|---|---|---|---|---|---|---|
| Bioactive | Gravure Print Treatment | | | Blade coating Treatment | | |
| paper | (Lysis measurement/mm) | | | (Lysis measurement/mm) | | |
| AG11 | Initial | 1 month | 2 months | Initial | 1 month | 2 months |
| AG20 | 31.4 | 34.6 | 30.3 | — | — | — |
| MS2 | — | — | — | — | — | — |
| T4 | — | — | — | — | — | — |

— Indicates no zones of lysis observed (phage not infective)

Phage titres after overnight storage of phage-based paper in buffer solution indicated that only gravure printed Salmonella phage AG11 diffused out of the bioactive paper (Table 15). Phage titres in buffer for all the other papers were below the 100 pfu/ml detection limit of the assay.

TABLE 15
Phage diffusion from printed and coated bioactive paper. Phage-
based paper was stored in 5 ml of phage buffer for 24-48 h at
room temperature then the phage titres in the buffer were determined
to investigate if phages diffused from the paper.

| | Phage titre (pfu/ml) - 24 h | | Phage titre (pfu/ml) - 48 h | |
|---|---|---|---|---|
| | Gravure | Blade | Gravure | Blade |
| Phage | Printing | Coating | Printing | Coating |
| AG11 | 2.5 × 10⁵ | ND | >10⁵ | ND |
| AG20 | ND | ND | ND | ND |
| MS2 | ND | ND | ND | ND |
| T4 | ND | ND | ND | ND |

ND—Not detected (below the detection limit of 100 pfu/ml)

### Thermal and Piezoelectric Inkjet Printing

### Ink Formulation and Inkjet Printing

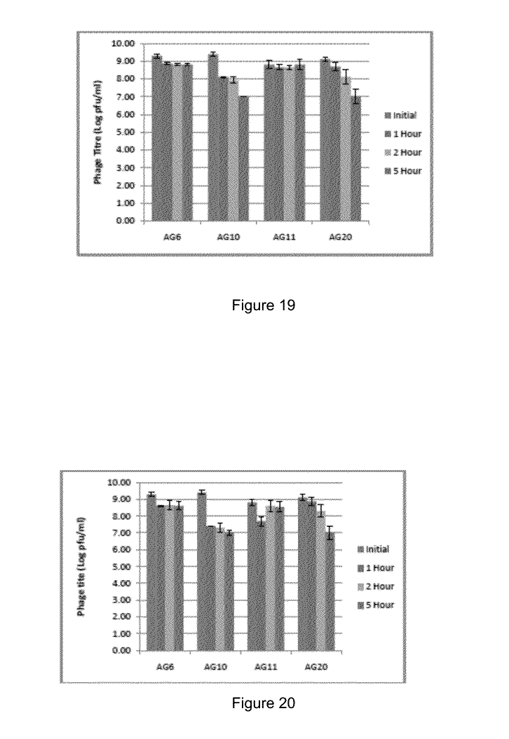

The effect of inks containing two different concentrations of glycerol (30% and 50%) on the infectivity of the phages was determined after five hours of exposure (FIG. 19 and FIG. 20). There was no significant difference (P >0.05) between the two glycerol concentrations on infectivity of phages AG6 and AG11. However, titres of phages AG10 and

AG20 declined by about 2 log₁₀ pfu/ml after five hours exposure to glycerol at both concentrations. The 30% glycerol concentration was selected for future piezo printing experiments.

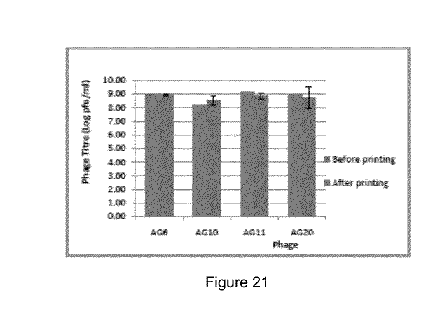

Phages were printed and the bio-ink was collected after printing to determine potential shearing effects by piezoelectric printing. Results indicated that there was no significant difference between the phage titres before and after printing (P >0.05) (FIG. 21).

Determination of Infectivity of Bioactive Paper and Phage Diffusion from the Paper

Phage infectivity was determined semi-quantitatively by the overlay technique and quantitatively after thermal and piezo inkjet printing on uncoated Whatman No. 1 paper. The infectivity of all of the phages, except AG11 was negatively affected by thermal inkjet printing as was evident by smaller or no zones of inhibition produced by the printed phage (Table 16). Paper containing phage AG6 produced barely noticeable zones of lysis when the phage was applied using both print techniques. In comparison application of paper containing phage AG11 paper prior to growth of the host bacterium on solid medium resulted in larger zones of inhibition compared to the other phages for both print methods while paper containing AG20 phage showed no infectivity (no zones of lysis) when applied by both printing methods.

TABLE 16
Infectivity of piezoelectric and thermal inkjet printed phage-based paper. Phage-based
paper was stored in phage buffer or dry at normal atmospheric conditions (about 50%
RH) or at about 80% RH for approximately 6 h. Infectivity of phage-based paper was
subsequently assessed by the overlay method.

| | Piezoelectric IJ Printing | | | Thermal IJ Printing | | |
|---|---|---|---|---|---|---|
| | | 50% RH | 80% RH | Storage in | 50% RH | 80% RH |
| | Storage in | Storage | Storage | buffer | Storage | Storage |
| | buffer | Diameter | Diameter | Diameter | Diameter | Diameter |
| | Diameter of | of | of | of | of | of |
| Phage | lysis/mm | lysis/mm | lysis/mm | lysis/mm | lysis/mm | lysis/mm |
| AG6 | +/− | +/− | +/− | +/− | +/− | +/− |
| AG10 | 26 | 27 | 27 | 26 | +/− | 26 |
| AG11 | 28 | 28 | 29 | 27 | 27 | 28 |
| AG20 | — | — | — | — | — | — |

— No inhibition observed
+/− Marginal (barely visible inhibition observed which could not be accurately measured.

After bioactive paper was placed in phage buffer for approximately six hours, only phage AG11 was observed to diffuse from uncoated Whatman No. 1 paper after application by both thermal and piezoelectric inkjet printing (Table 17). Approximately 1% of AG11 phages printed by piezo and 0.002% of phages immobilized by thermal printing leached from the paper.

TABLE 17
Phage diffusion from phage-based paper after inkjet printing.
Phage-based paper was stored in 5 ml of phage buffer for about
6 h at room temperature then the phage titres in the buffer were
determined to investigate if phages diffused from the paper.

| | Phage titre of diffused phages (pfu/ml) | |
|---|---|---|
| Phage | Piezoelectric IJ Printing | Thermal IJ Printing (pfu/ml) |
| AG6 | ND | ND |
| AG10 | ND | ND |
| AG11 | 1.2 × 10⁴ | 2.0 × 10² |
| AG20 | ND | ND |

ND—Not detected

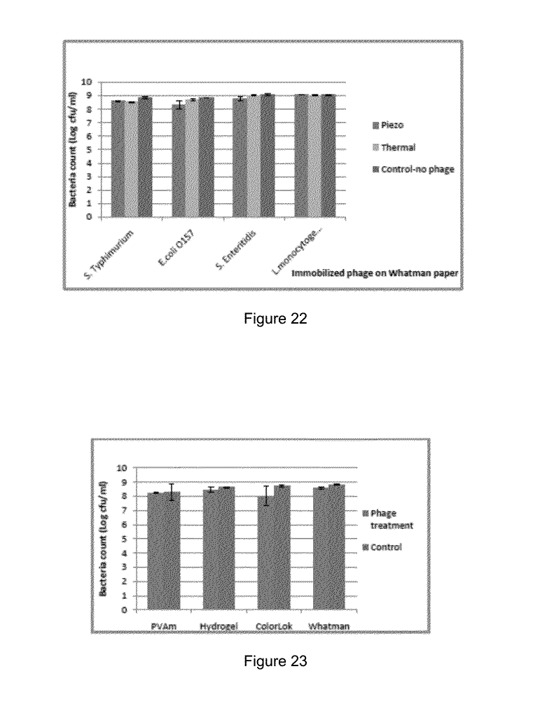

Quantitative results from the broth experiment indicated the efficacy of the bioactive paper for the control of their hosts. However, phage-based Whatman paper did not result in significant reductions of host bacteria after phage application by either inkjet printing technique (P >0.05; FIG. 22). A reduction in count of 0.33 log₁₀ cfu/ml of S. Enteritidis was the highest achieved and this was produced following deposition of phage AG11 onto paper by piezoelectric printing (P=0.055). Application of the other phages to paper by this technique resulted in reductions in counts of 0.24, 0.24 and 0.002 log₁₀ cfu/ml for phage/host combinations of S. Typhimurium/AG6, E. coli 0157:H7/AG10 and L. monocytogenes AG20, respectively.

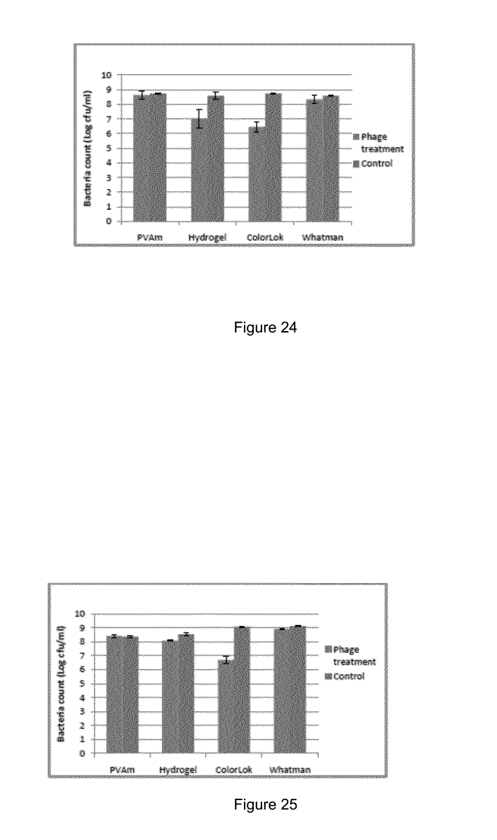

Phages were also printed on cationic PVAm, Hydrogel and ColorLok coated papers by piezoelectric inkjet printing. The phage-based ColorLok papers resulted in significant reductions in counts of 0.69 log₁₀ cfu/ml for S. Typhimurium by phage AG6 (FIG. 23), 2.28 log₁₀ cfu/ml for E. coli 0157:H7 by phage AG10 (FIGS. 24) and 2.35 log₁₀ cfu/ml for S. Enteritidis by phage AG11 (FIG. 25) (P<0.05). The AG10 phage-based hydrogel paper achieved a reduction in count of 1.58 log₁₀ cfu/ml for E. coli 0157:H7 (FIG. 24). However, no significant reduction in count for L. monocytogenes was observed using any of the AG20 phage-based papers (P>0.05) (FIG. 26).

### Piezoelectric Printing on Cationic ColorLok Paper

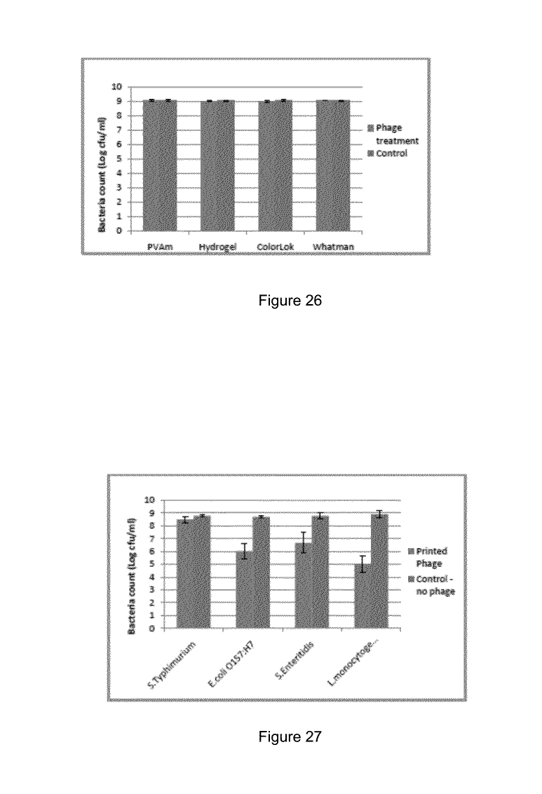

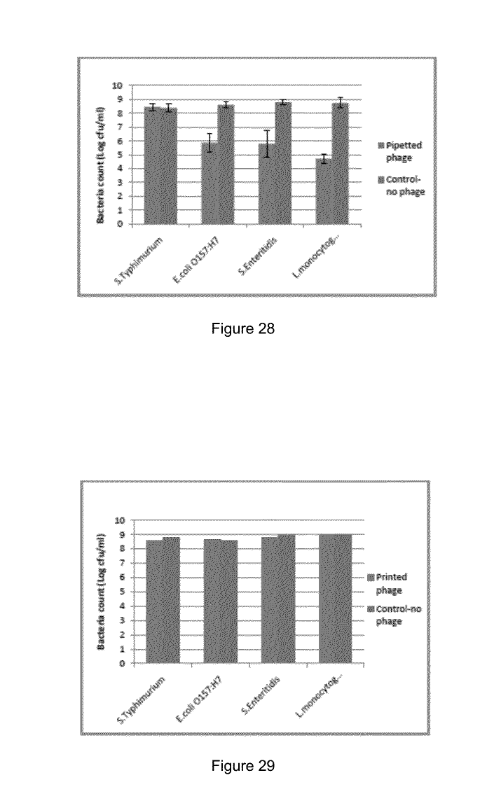

Phage printed on ColorLok paper produced higher reductions in counts of their host bacteria than phage printed on the other types of paper tested (FIGS. 23, 24, 25 and 26). Thus, ColorLok paper was used to study the effect of piezoelectric inkjet printing on phage infectivity. Significant differences in bacterial growth were observed between cultures incubated with ColorLok paper containing no phage and three of the printed phages, AG10, AG11 and AG20. Significant reductions in count (P<0.05) of 2.68 log₁₀ cfu/ml for E. coli 0157:H7, 2.11 log₁₀ cfu/ml for S. Enteritidis and 3.89 log₁₀ cfu/ml for L. monocytogenes were observed when cultures were grown in the presence of printed paper containing phage AG10, AG11 and AG20, respectively (FIG. 27). Phage AG6 was only capable of reducing counts by 0.28 log₁₀ cfu/ml for S. Typhimurium. The effect of delivery of higher phage titres on the paper was investigated. The Dimatix printer was estimated to deliver 25 pl to each 4.9 cm² area of paper. When 100 pl of the ink containing phage was delivered onto the same paper type by pipette to cover the same surface area, similar reductions in count were obtained; being 2.74 log₁₀ cfu/ml for E. coli 0157:H7, 3.01 log₁₀ cfu/ml of S. Enteritidis and 4.03 log₁₀ cfu/ml for L. monocytogenes and no reduction was observed using AG6 phage-based paper (FIG. 28).

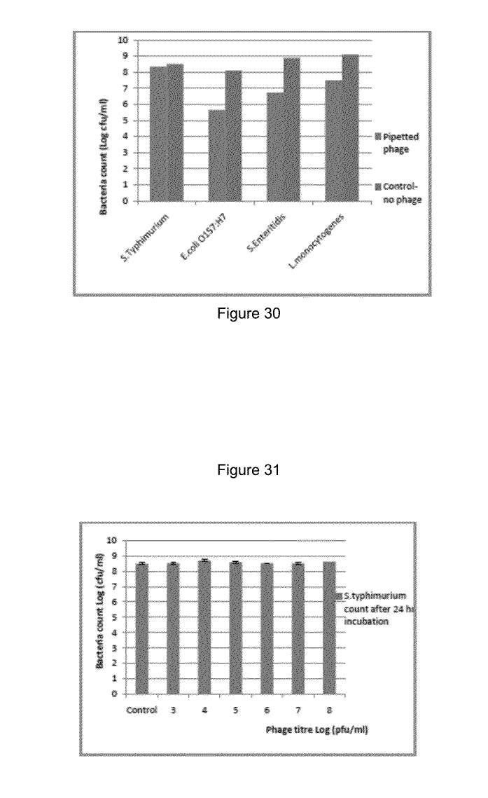

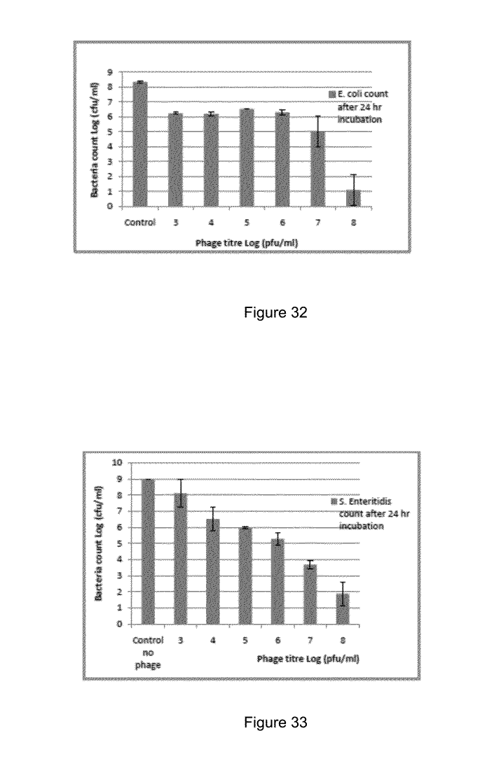

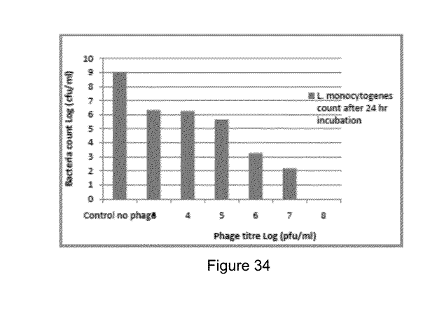

Stability of phage-based paper is critical for the successful incorporation of phages into packaging material. To assess stability of the phage deposited onto ColorLok paper, the phage-containing papers were stored for one month at 20-25° C. and 80-85% RH on 2 separate occasions. The phage printed on ColorLok paper was not stable, since no reduction in counts of the relevant host bacterium was observed when they were grown in the presence of the stored paper (FIG. 29). However, based on the average counts from the 2 separate experiments, the paper containing phage deposited by pipetting retained some infectivity after storage and resulted in reductions in count of 2.45 log₁₀ cfu/ml for E. coli 0157:H7, 2.16 log₁₀ cfu/ml for S. Enteritidis and 1.62 log₁₀ cfu/ml L. monocytogenes using paper containing AG10, AG11 and AG20 phage, respectively (FIG. 30). A reduction in count of 0.23 log₁₀ cfu/ml for S. Typhimurium was observed with papers containing AG6.

### Theoretical Estimate of Phages Delivered to ColorLok Paper by Piezoelectric Printing and Pipettinq

The Dimatix piezoelectric inkjet printer nozzle delivered 10 pl drop volumes. It was estimated that the printer deposited 500 drops/cm². Thus, for the paper size used in these experiments (2.5 cm diameter) it can be calculated that 24.5 μl of ink were deposited on the paper surface. As the initial phage titres in the ink are known, the number of phage present in 24.5 μl and 100 μl can be calculated (Tables 18 and 19).

TABLE 18
Estimate of number of phage particles delivered by
piezoelectric inkjet printing on ColorLok paper

| | | Bio-ink | | |
|---|---|---|---|---|
| | Initial phage | phage titre | Phage Titre | Phage |
| Phage | titre pfu/ml | pfu/ml | pfu/24.5 μl | Titre/cm² |
| AG6 | 4.50E+09 | 3.00E+08 | 7.35E+06 | 1.50E+06 |
| AG10 | 5.00E+08 | 7.00E+07 | 1.72E+06 | 3.50E+05 |
| AG11 | 1.00E+09 | 3.50E+08 | 8.58E+06 | 1.75E+06 |
| AG20 | 1.00E+09 | 3.00E+08 | 7.35E+06 | 1.50E+06 |

TABLE 19
Estimate of number of phage particles delivered
by pipetting on ColorLok paper

| | | Bio-ink | | |
|---|---|---|---|---|
| | Initial phage | phage titre | Phage Titre | Phage |
| Phage | Titre pfu/ml | pfu/ml | pfu/100 μl | Titre/cm² |
| AG6 | 4.50E+09 | 3.00E+08 | 3.00E+07 | 6.12E+06 |
| AG10 | 5.00E+08 | 7.00E+07 | 7.00E+06 | 1.43E+06 |
| AG11 | 1.00E+09 | 3.50E+08 | 3.50E+07 | 7.14E+06 |
| AG20 | 1.00E+09 | 3.00E+08 | 3.00E+07 | 6.12E+06 |

Experimental Estimate of Phages Printed by Piezoelectric Printing onto ColorLok Paper

Phage AG11 was printed onto ColorLok paper and subsequently the DNA was extracted and the approximate amount of AG11 DNA on the paper was determined by real-time PCR. Results obtained from the Nanodrop 1000 spectrophotometer (Thermo Scientific) indicated that approximately 351.1 μg/μl of DNA was extracted from the 4.9 cm² paper sample. From the real-time PCR analysis an estimated 6.63 log₁₀ pfu/cm² of AG11 was deposited on the paper, which is comparable to the estimated value (Table 18).

The approximate amount of infective phages on the phage-based paper was determined by comparing bacterial counts achieved in the presence of the phage-printed paper (FIGS. 2.12, 2.13, 2.14, 2.15) with those obtained in the presence of free phage (FIGS. 2.16, 2.17, 2.18, 2.19). E. coli 0157:H7, S. Enteritidis and L. monocytogenes counts in the presence of printed phages and tested within one day of preparation were similar to those obtained with 10⁶-10⁷ pfu/ml of free AG10, 10⁴-10⁵ pfu/ml of free AG11 and 10⁵-10⁶ pfu/ml of free AG20. Similar results were obtained for the pipetted phages. The printed phage-based paper was not infective after one month storage at room temperature at 80-86% RH (FIG. 29). However, the paper on which phages were deposited by pipette retained infectivity for 3 of the 4 phages. Logo reductions in count of E. coli 0157:H7 and S. Enteritidis by phage-based papers produced by pipetting indicated approximately 10⁶-10′ pfu/ml of infective AG10 and 10³-10⁴ pfu/ml of infective AG11. Results for pipetted AG20 phage after storage were not reproducible for the two experimental replicates conducted since one experimental replicate indicated no host reduction and the second replicate demonstrated L. monocytogenes reduction similar to the initially pipetted phage suspension, which resulted in a reduction in counts of 3.17 log₁₀ cfu/ml, which correlated to approximately 10⁵-10⁶ pfu/ml of infective AG20.

Using phages immobilized within packaging material can provide a convenient, and cost effective method to control pathogens during food storage. Phage-based packaging material was developed during this study using an immobilization approach that can be used for any phage and does not require chemical or genetic phage modification. Rather the printing technology used would provide the opportunity to produce phage-based antibacterial packaging on a commercial scale. Immobilization of phages from the Leviviridae, Siphoviridae and Myoviridae families was performed by blade coating and gravure printing. However, the titre of MS2, the Leviviridae phage, was observed to decline within three months of storage at 4° C. Since this reduced activity was not representative of the stability of the phages in the culture collection the immobilization of this phage by inkjet printing was not investigated in this example. Since tailed phages are the most predominant group of phages in existence, with the Siphoviridae and Myoviridae being the most abundant (Ackermann, 2006), bacteriophages from these two families were used for inkjet printing.

Successful immobilization of phages by printing or coating requires that these processes should not have a negative impact on phage survival, either by the formulated inks or by the technologies employed. Blade coating is commonly used for commercial application of paper coatings while gravure printing is used for high quality print applications. These technologies could potentially be modified for application of phages to paper surfaces. Inkjet printing technologies were also explored as another universal phage immobilization method. Both blade coating and gravure printing were anticipated to provide high throughput production of phage-based packaging. However, only one phage, Salmonella AG11, was stable under the conditions required for these coating and printing techniques. The fact that a higher AG11 phage titre was initially used could be a contributory factor for the demonstrated stability compared to the other phages. It is also possible that thermal inactivation of the other phages occurred during the blade coating process since a holding temperature of 45° C. was used for the Bioink prior to coating and also a temperature of 66° C. for 30 sec was used for drying of the phage coating. Previously phage AG11 was observed to withstand the highest storage temperature of 42° C. for 24 h with less than 1 log₁₀ pfu/ml reduction in titre (Anany, 2010). This heat resistance may have contributed to the ability of phage AG11 to survive these printing and coating processes. The porosity of the paper used for printing could also be another factor preventing phage infectivity from being observed for all of the phages except AG11, since entrapment of phages within the paper would prevent phages from being able to adsorb to their host bacterium. Blade coating and gravure printing of T4 phages on paper with different air permeabilities led to similar observations by Jabrane et al. (2011). Infectivity of the phage-based paper printed using inkjet printing also indicated that the phages were probably negatively affected by thermal inkjet printing, except for phage AG11.

The estimated amount of phages deposited onto the paper by double layer thermal inkjet printing was approximately 100-fold more than that deposited by the piezoelectric method. This is due to the lower dilution factor of the thermal inkjet bio-ink used and also since the drop size of piezo printing is 10 pl compared to 25 pl for thermal printing. Based on the higher volume of phages delivered to the paper it was expected that thermal inkjet printed phage-based paper would exhibit a significantly increased infectivity compared to phages that were subjected to piezo printing, however, the opposite was observed. Thermal inkjet printing was successfully used for printing of enzymes (Di Risio and Yan, 2007, 2008, Lonini et al., 2008), cells (Xu et al., 2005) and other biomolecules. However, high temperatures of 300° C. for milliseconds are used for ink ejection during this printing process (Kipphan, 2001). It is possible that the phage activity was negatively affected by heating of the bio-ink for this printing technique or a combination of thermal inactivation and entrapment of phages in the pores of the uncoated Whatman paper may have resulted in reduced infectivity. Piezolelectric inkjet printing was therefore identified as having the greater commercial potential.

Modification of inks, equipment and the printing process may be necessary to ensure successful printing of the phages. The biological inks used for inkjet printing in this experiment were formulated to provide optimum ink ejection during printing, while promoting phage survival. Trition X-100 is a non-ionic surfactant, which does not have a significant effect on enzyme activity (Di Risio and Yan, 2010) and glycerol is compatible with proteins (Hossain et al., 2009). Glycerol was also added to the bio-ink for its humectant properties to reduce the susceptibility of the phages to desiccation during storage. Bio-inks composed of 30% and 50% glycerol were observed to have a similar effect on the phages, though reductions in the titre of AG10, the E. coli 0157:H7 phage and AG20, the L. monocytogenes phage, occurred within 5 hours. However, it was anticipated that this could be a transient effect since the inks were expected to diffuse into the paper substrate and allow phages to be retained on the paper surface when using coated paper with small pore sizes; potentially having a reduced effect on the phages. Future experiments demonstrated that when phages AG10 and AG20 were printed they were able to reduce counts of their host bacterium by 2.68 and 3.89 log₁₀ cfu/ml, respectively; hence permanent inactivation of the phages did not occur.

Food contamination is likely to occur on the surface of food unless they are ground, when contamination will be found throughout the product. Direct contact of phage-based packaging material with food would therefore result in infection of the host bacterium, if present, followed by phage replication and lysis of the host cells with the release of progeny phages into the food, where they can potentially diffuse and initiate other infection cycles. Additionally, if phages diffuse out of the packaging material they would not require host infection to disperse throughout the food. Based on the results of both gravure printed and inkjet printed phages, it was observed that about 1% of the Siphoviridae phage AG11 was able to diffuse out of the uncoated Whatman paper, unlike the other phages. The reason for this phage being the only one able to diffuse out from the paper is not clear. Experiments to observe phage diffusion from the paper were only conducted during initial printing on Whatman paper and were not conducted with coated papers. It is uncertain whether there could be additional interaction of the phages with other cationic papers, which influences their ability to diffuse out of the paper. Additional, studies would have to be conducted with the packaging materials to determine the likelihood of phage diffusion into the food product. It is anticipated that low levels of phages may be released into the product unless infection of the host bacterium occurs. Although some consumers may be willing to pay more for phage treated foods based on their socioeconomic status and consumer awareness of the technology (Yeboah et al., 2012), the presence of phages in the packaging rather than their direct application to the food product may be more acceptable to consumers who may be sceptical of the use of phages in foods. Infectivity testing of the inkjet printed Whatman phage-based paper indicated no significant reduction of host bacteria. This may possibly be attributed to penetration of the phages within the 11 μm diameter pores of this absorbent paper and possible electrostatic interaction of the positively charged tail fibres with the negatively charged cellulose fibres. Piezoelectric printing of horseradish peroxidase on coated and increased sizing of papers contributed to biological inks being retained at the paper surface (Di Risio and Yan, 2008). Also, Jabrane et al. (2009) observed that the use of cationic-coated paper for gravure printing of T4 phages resulted in enhanced phage infectivity. Similar observations were made during this experiment when the phage printed on cationic ColorLok paper resulted in higher bacterial reductions compared to phage printed onto the uncoated Whatman paper. This observation is most likely due to the phages being retained at the paper surface, therefore facilitating interaction with host bacterium compared with the other types of paper. Cationic hydrogel treated paper containing phage AG10 was the only other bioactive paper that resulted in significant reductions in count of the host bacterium. Washing off excess PVAm and hydrogel polymers after coating the Whatman No. 1 paper may have contributed to these papers having an uneven surface distribution of the polymers and to being more porous than the ColorLok.

Oriented phage immobilized on the paper is critical for adsorption to host cell receptors. Measurement of the infectivity of the phage-based paper compared to the infectivity of similar concentrations of free phages could give an indication of the number of phages that are randomly oriented or entrapped in the paper pores following immobilization. The DNA of AG11 phage printed on ColorLok paper was extracted and the quantity of printed phages was determined by qPCR. Results obtained were comparable with estimates of numbers of phage particles deposited on the surface of the paper based on initial titres in the ink. Also different dilutions of free phage were added to 10³ cfu/ml of the appropriate host to estimate the effect of the phages. The resulting host bacterial count after addition of free phages was used to estimate the infectivity of the phage-based paper. Theoretical estimates of the amount of printed phages correlated with the estimated number of infective AG10 and AG20 phages. The slightly lower infectivity of phage AG11 could probably be attributed to experimental variability or differences in the phage-host interactions. Additionally, it was observed that free phage AG6 was unable to significantly reduce populations of its host, S. Typhimurium, even when 10⁸ pfu/ml of phage were used. These results could explain previous results where AG6 phage-based paper did not result in significant reduction of its host. A higher frequency of development of resistant host bacteria was observed with AG6 phage and its host, S. Typhimurium compared to other tested Salmonella phages and their hosts (Anany, 2010). This phage would therefore not be ideal for use in biocontrol and is not reflective of the effect of the printing immobilization processes.

Phage-based packaging material is anticipated to provide control of food-borne pathogens during storage; therefore the stability of the developed paper is crucial. However, the paper printed with phage was not stable after one-month storage. An increase of approximately four times the amount of bio-ink pipetted onto the paper did not result in a significantly higher amount of phages delivered to the paper compared to printing. However, the increased amount of glycerol in the ink prevented the paper from drying completely, which may have contributed to the increased stability of this pipetted phage-based paper after one month with activity slightly lower than that of freshly printed phage-based paper. This was true for all phages except AG20. Non-reproducible results were obtained between the two experimental replicates using pipetted AG20 phages, for which one experiment demonstrated no reduction in counts of L. monocytogenes while the other experiment resulted in reduction in counts of 3.17 log₁₀ cfu/ml. It is possible that non-homogenous phage delivery to the paper may have caused these results. Previous studies have established that use of high titres of phages was more successful for biocontrol and also phage cocktails decreased the possibility of development of phage resistance (Kudva et al., 1999, Hooton et al., 2011). Additionally, the phages used for biocontrol should be stable under different conditions such as temperature, low pH, and elevated salt concentrations. Phages AG11 and AG20 have demonstrated activity at lower temperatures though AG11 exhibited activity over a wider range of pH (4.5-7) and was unaffected by 5% NaCl (Anany, 2010). Optimizing inkjet printing parameters is necessary for successful phage printing. The nozzle of the piezoelectric printer was prone to clogging and there was a possibility that shear forces may be exerted on the phages. Frequent wash steps were therefore necessary during phage printing to prevent clogging and the piezoelectric printing process was very slow compared to the thermal inkjet printing. Printing of a 12 cm×18 cm paper surface area required approximately 20 min using the laboratory scale piezo printer. However, the likelihood of increased printing efficiency of commercial piezoelectric printers needs to be determined. Alternatively, other non-contact, non-thermal coating or printing technologies may potentially improve the efficiency of production of commercial phage-based packaging.

Reduction in contamination of the phage-based paper was observed during this experiment by the use of inkjet printing compared to blade coating and gravure printing. Inkjet printing of phages involves the spraying of the bio-ink onto the paper surface. However, blade coating and gravure printing techniques require direct contact of printing cylinders with the paper surface and therefore increases the chances of contamination being introduced, as was evident during this study. Good manufacturing practices should be implemented for the production of biologically active packaging materials and non-contact printing techniques would contribute to reduced contamination.

We conclude that piezoelectric inkjet printing provides an effective universal immobilization strategy for the commercial production of phage-based antimicrobial packaging materials. However, other non-contact, non-thermal printing or coating strategies may also have potential. The choice of printing substrate is important for retaining phages on the surface of the packaging material to facilitate control of their hosts. The potential exists for using other materials for phage printing, for example, other cationic, coated paper-based materials and edible films, which are currently used as sausage casings, and other films used for food packaging. This study establishes that delivery of an increased number of phages to the paper in combination with humectants, such as glycerol, improves stability of the printed phage-based paper. Printing of phage preparations with high titres and phage cocktails is also expected to provide more effective packaging material.

### Example 6

### Printing/Immobilization of Salmonella Newport Phage on Paper and Demonstrated Effectiveness using Chicken

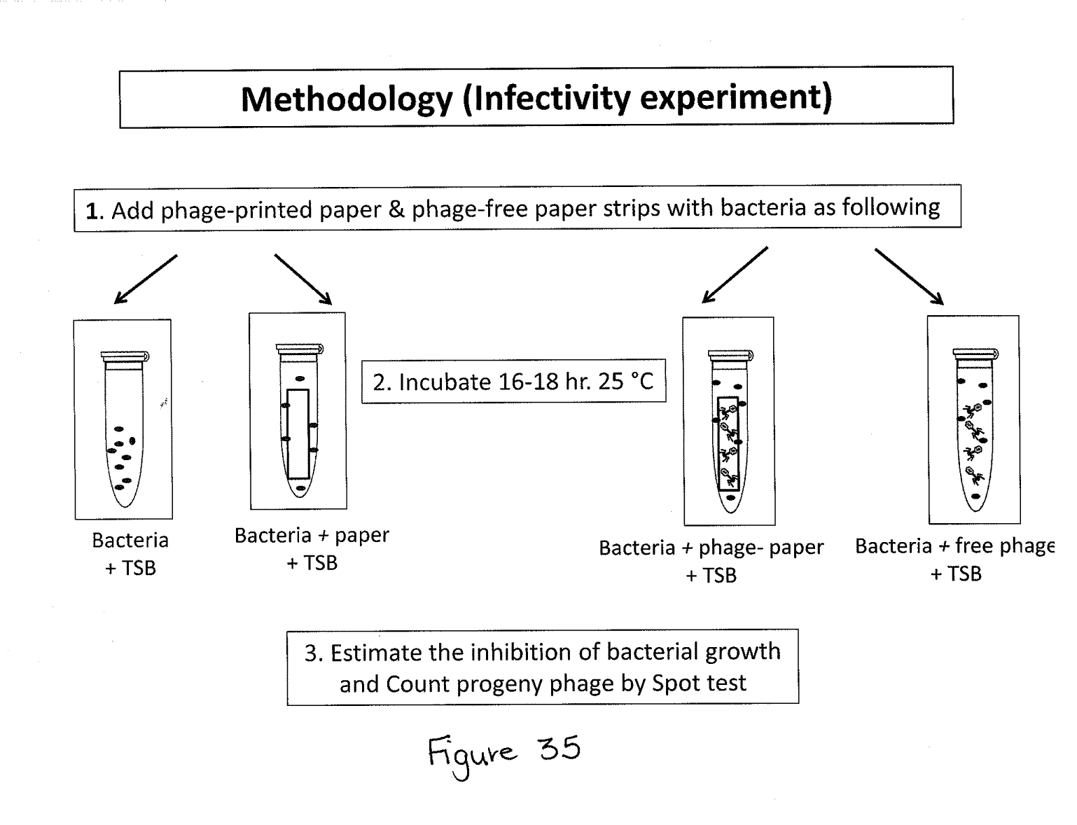

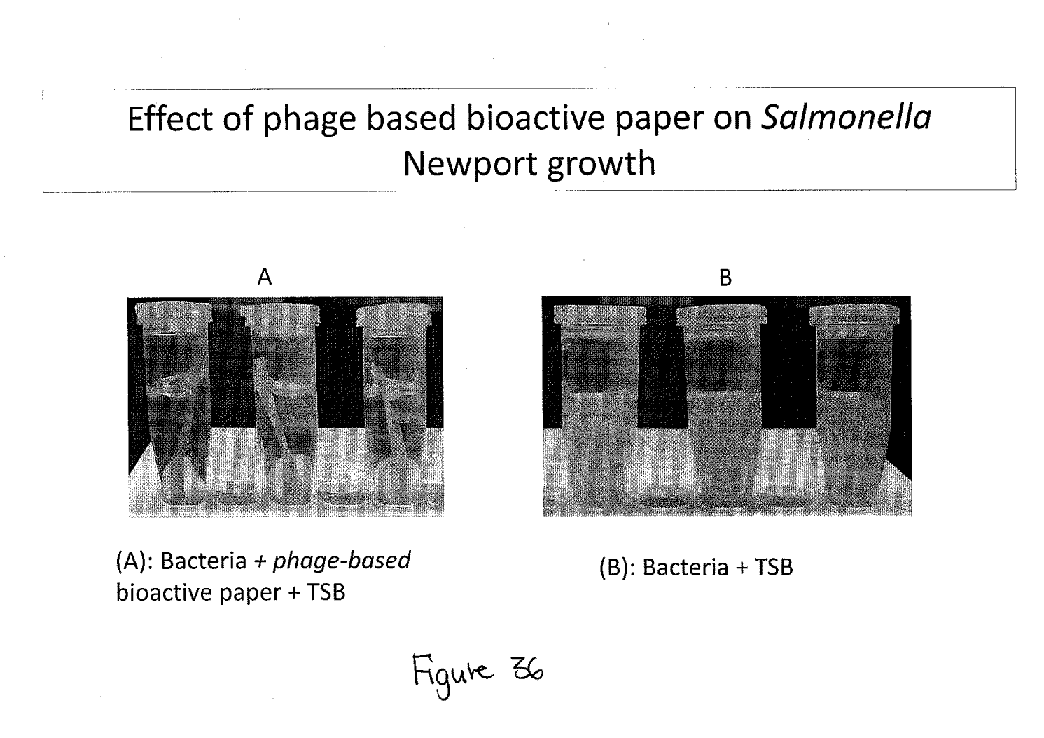

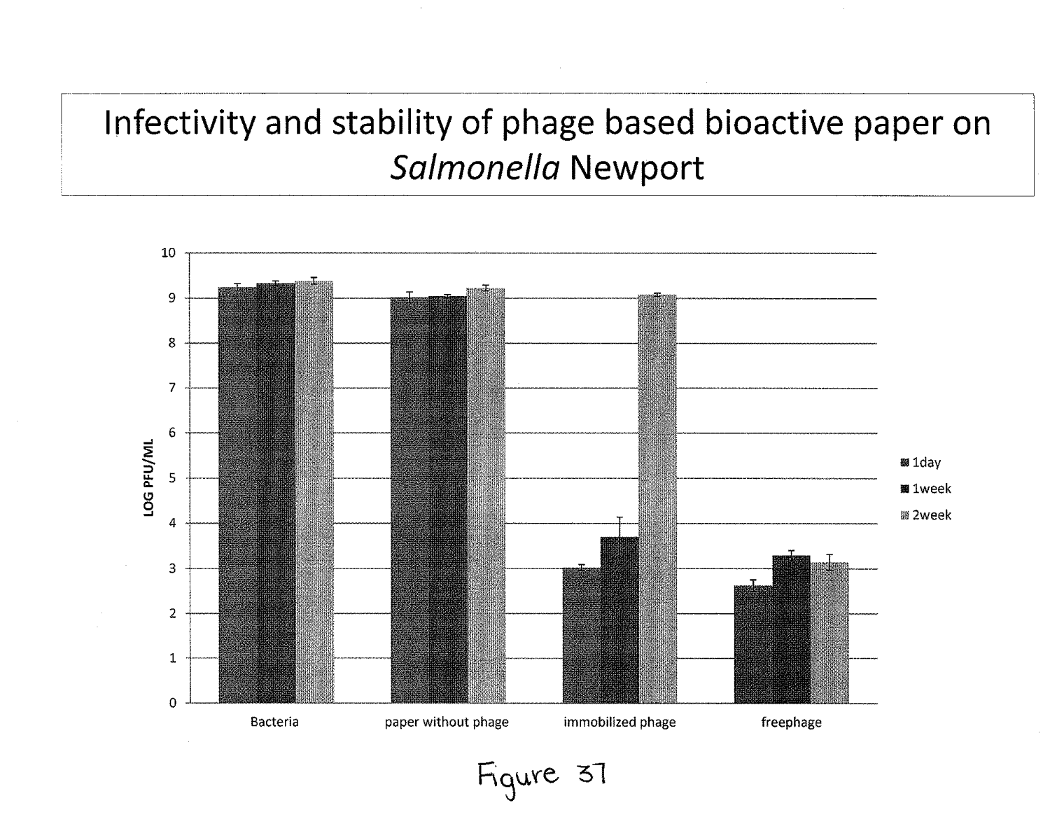

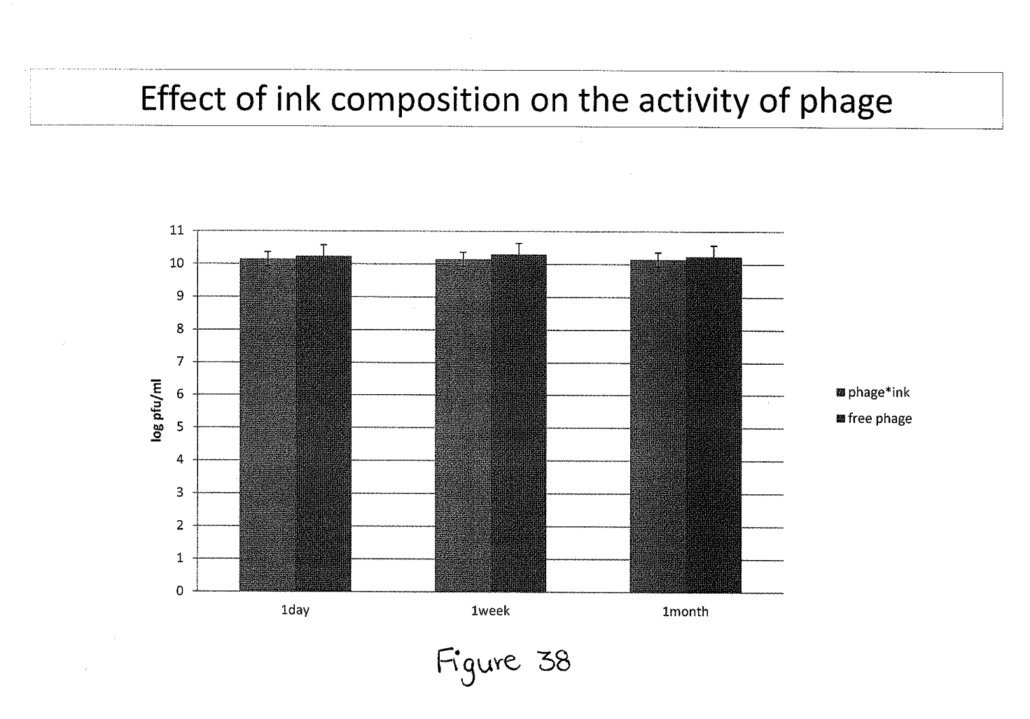

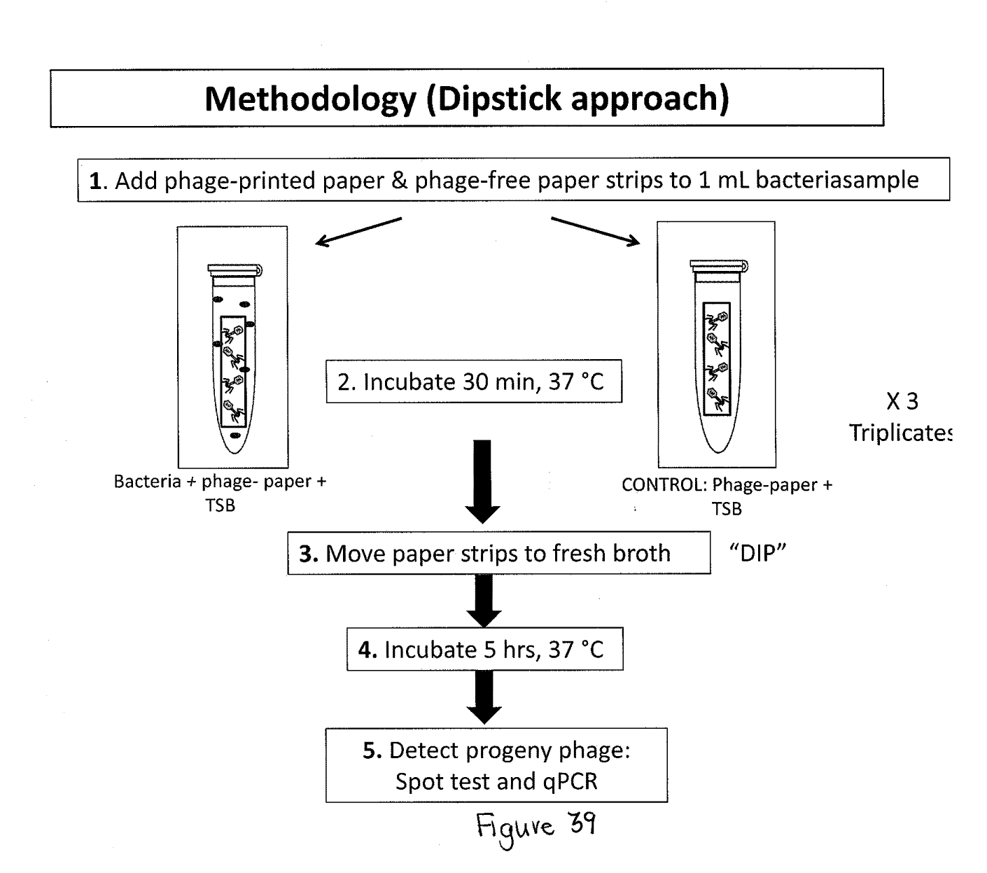

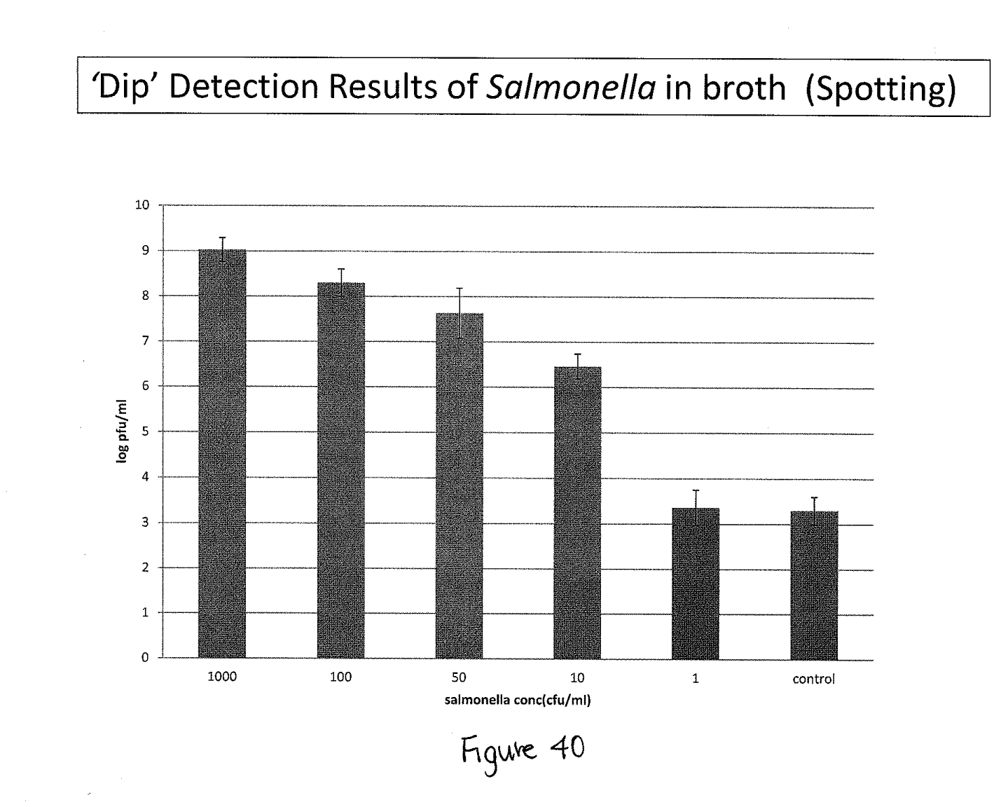

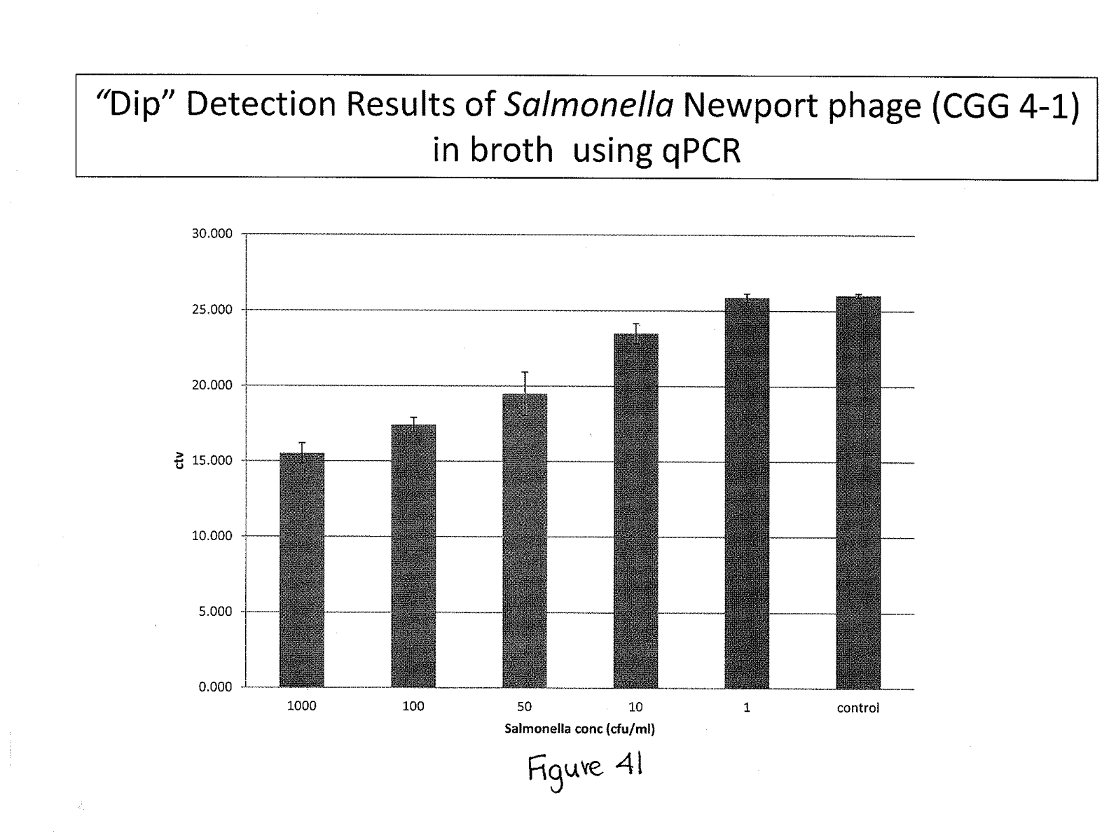

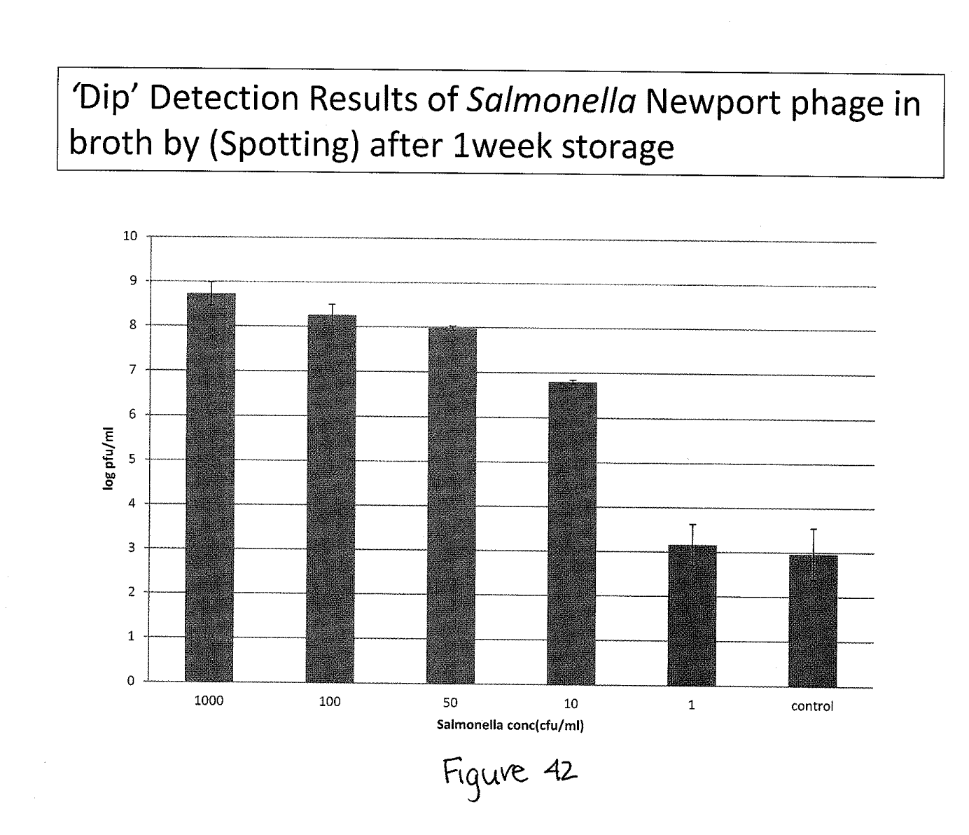

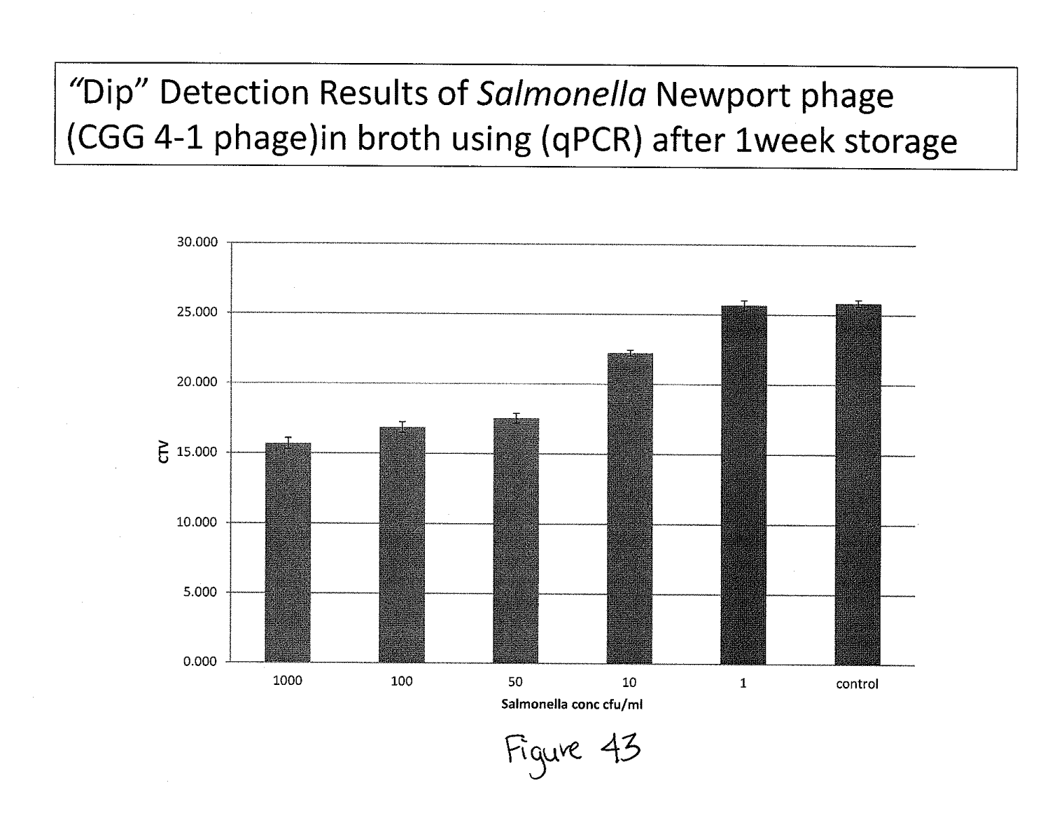

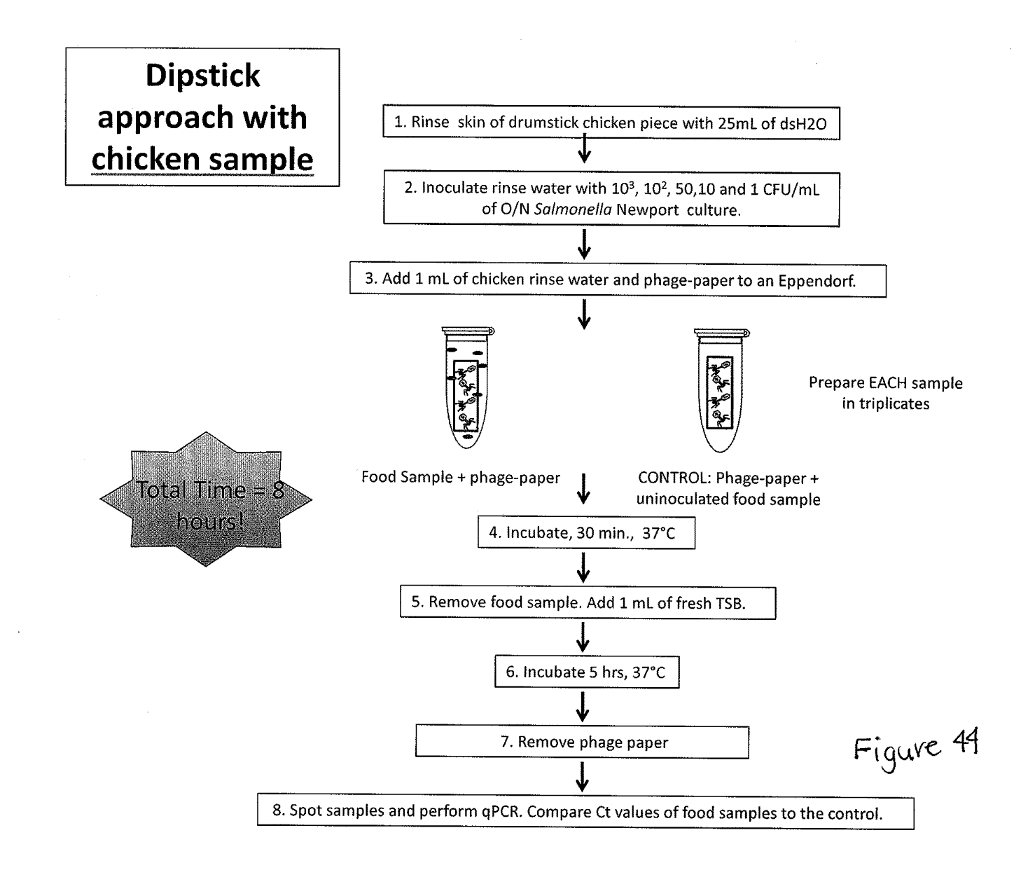

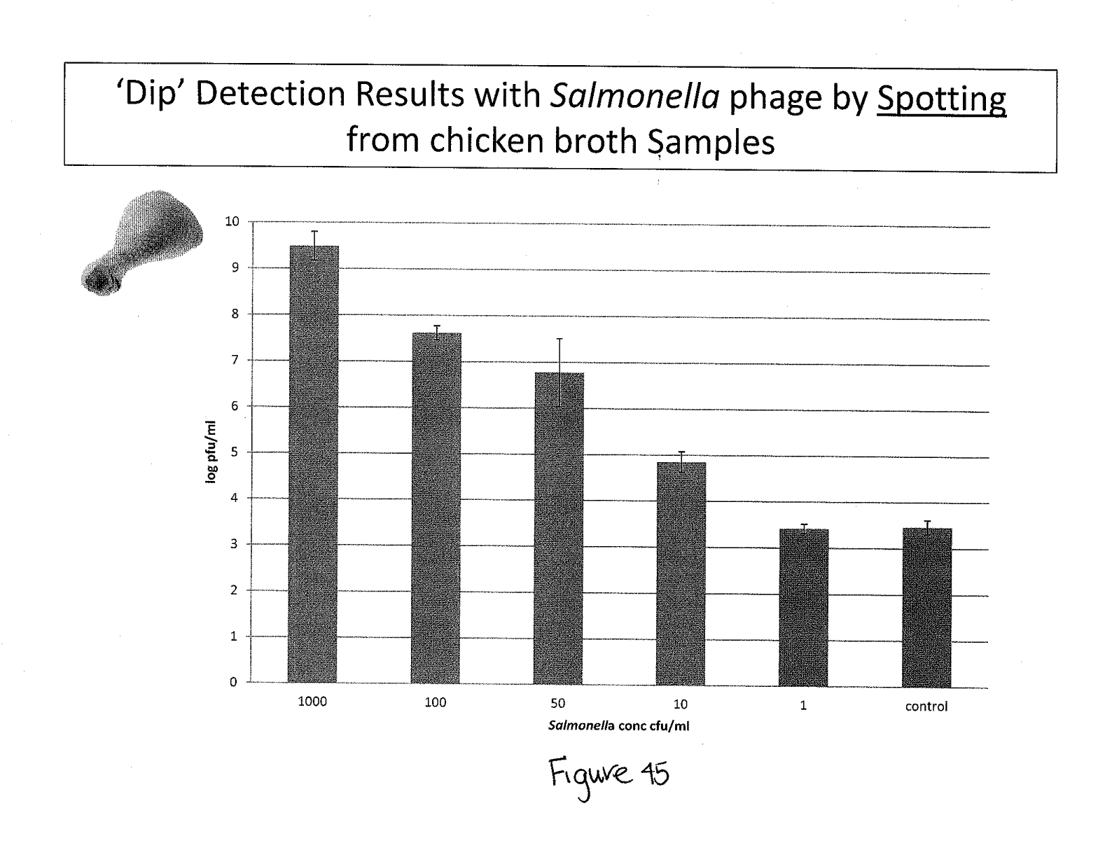

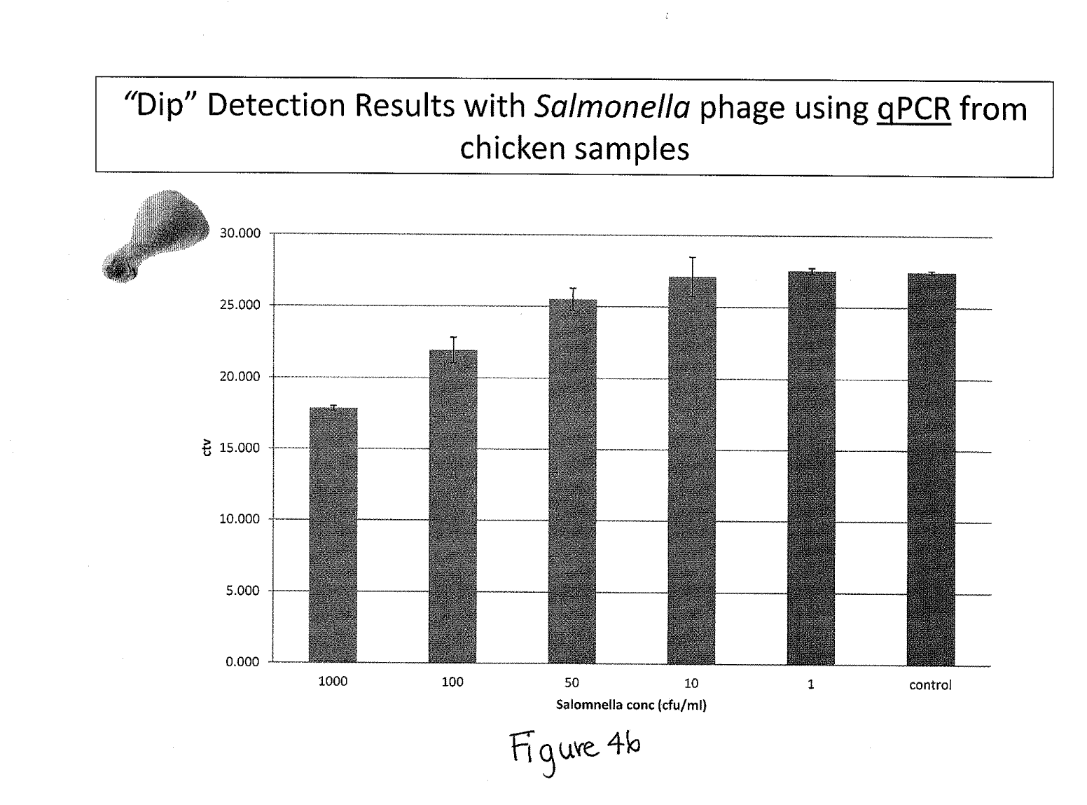

Briefly, the preparation of bio-inks and printing onto ColorLok™ paper are as set forth in the preceding examples incorporating Salmonella Newport phage (CGG 4-1 phage) in order to detect and control the growth of Salmonella Newport. Statistical analysis is also done as set forth in the preceding examples. The infectivity trials were done to determine the effect of piezoelectric printing on the phage. Detection protocols are as shown in FIGS. 35, 36, 39 and 44. In FIG. 44, a chicken piece was actually used to demonstrate that the invention does indeed work on food, that is, meat-specifically chicken in this example. This example results as shown below and in the noted figures demonstrate the efficacy of the bioactive paper for the control of salmonella.

TABLE 20
Detection Results of CGG 4-1 in Broth
Limit of detection is approximately 10 CFU/ml.
Values are significantly different from the control
(phage-printed paper in TSB).

| Approximate | CGG4-1 | log | | | |
|---|---|---|---|---|---|
| Bacteria Count | Titre Log | (pfu/mL) | Ct | Ct | |
| (CFU/mL) | (PFU/mL) | SD | Mean | SD | P- value |
| 1000 | 9.02 | 0.26 | 15.52 | 0.67 | 1.17E−07* |
| 100 | 8.30 | 0.3 | 17.42 | 0.48 | 1.49E−07* |
| 50 | 7.63 | 0.55 | 19.49 | 1.42 | 8.77E−05* |
| 10 | 6.46 | 0.27 | 23.49 | 0.66 | 0.00013* |
| 1 | 3.35 | 0.39 | 25.86 | 0.27 | 0.35 |
| Control | 3.30 | 0.30 | 26.01 | 0.12 | |

Table 20 shows the detection of Salmonella Newport in broth (TSB) using phage-printed paper (Dipstick approach) of FIG. 39. The progeny phages were counted (PFU/mL) using overlay technique and detected using RTPCR (Ct values). Data are from the mean of three replicates. Detection limit was around 10 CFU/mL.

TABLE 21
Detection Results of CGG 4-1 in broth after 1 week storage.
Limit of detection is approximately 10 CFU/ml.
*Values are significantly different from the control
(phase-printed paper in TSB).

| Approximate | CGG4-1 | log | | | |
|---|---|---|---|---|---|
| Bacteria Count | Titre Log | (pfu/mL) | Ct | Ct | |
| (CFU/mL) | (PFU/mL) | SD | Mean | SD | P- value |
| 1000 | 8.72 | 0.26 | 15.68 | 0.38 | 9.64E−09* |
| 100 | 8.25 | 0.24 | 16.68 | 0.37 | 3.43E−08* |
| 50 | 7.98 | 0.05 | 17.53 | 0.35 | 2.87E−08* |
| 10 | 6.80 | 0.03 | 22.23 | 0.19 | 5.31E−07* |
| 1 | 3.16 | 0.46 | 25.67 | 0.35 | 0.22 |
| Control | 2.96 | 0.57 | 25.84 | 0.23 | |

Table 21 shows the detection of Salmonella Newport in broth (TSB) using phage-printed paper stored for one week at room temperature with approximately 80% RH (Dipstick approach). The progeny phages were counted (PFU/ml) using overlay technique and detected using RTPCR (Ct values). Data are from the mean of three replicates. Detection limit was around 10 CFU/ml.

TABLE 22
Detection Results of CGG 4-1 in Chicken Sample
Detection Results of CGG 4-1 in Chicken sample

[symbol]

| Approximate | CGG4-1 | log | | | |
|---|---|---|---|---|---|
| Bacteria Count | Titre Log | (pfu/mL) | Ct | Ct | |
| (CFU/mL) | (PFU/mL) | SD | Mean | SD | P- value |
| 1000 | 9.48 | 0.31 | 17.85 | 0.16 | 8.47E−10* |
| 100 | 7.61 | 0.15 | 21.93 | 0.89 | 1.52E−05* |
| 50 | 6.77 | 0.74 | 25.50 | 0.76 | 0.001* |
| 10 | 4.75 | 0.37 | 16.95 | 1.13 | 0.34 |
| 1 | 3.41 | 0.10 | 27.55 | 0.19 | 0.42 |
| Control | 3.45 | 0.15 | 27.45 | 0.10 | |

Limit of detection is approximately 50 CFU/mL.
Values are significantly different from the control

This table shows the detection of Salmonella Newport in chicken using phage-printed paper (Dipstick approach). The progeny phages were counted (PFU/mL) using overlay technique and detected using RTPCR (Ct values). Data are from the mean of three replicates. Detection limit was around 50 CFU/mL.

### REFERENCES

ALFADHEL, M., PUAPERMPOONSIRI, U., FORD, S. J., MCINNES, F. J. & VAN DER WALLE, C. F. 2011. Lyophilized inserts for nasal administration harboring bacteriophage selective for Staphylococcus aureus: In vitro evaluation. International journal of pharmaceutics, 416, 280-287.
ALVAREZ-GONZALEZ, E., ALFADHEL, M., MANE, P., FORD, S. J., MOORE, B. D. & VAN DER WALLE, C. F. 2012. Bioprocessing of bacteriophages via rapid drying onto microcrystals. Biotechnology progress, 28, 540-548.
ANANY, H., CHEN, W., PELTON, R. & GRIFFITHS, M. W. 2011. Biocontrol of Listeria monocytogenes and Escherichia coli 0157:H7 in Meat by Using Phages Immobilized on Modified Cellulose Membranes. Applied and Environmental Microbiology, 77, 6379-6387.
ARCHER, M. J. & LIU, J. L. 2009. Bacteriophage T4 Nanoparticles as Materials in Sensor Applications: Variables That Influence Their Organization and Assembly on Surfaces. Sensors, 9, 6298-6311.
BALASUBRAMANIAN, S., SOROKULOVA, I. B., VODYANOY, V. J. & SIMONIAN, A. L. 2007. Lytic phage as a specific and selective probe for detection of Staphylococcus aureus—A surface plasmon resonance spectroscopic study. Biosensors & Bioelectronics, 22, 948-955.
BENNETT, A. R., DAVIDS, F. G. C., VLAHODIMOU, S., BANKS, J. G. & BETTS, R. P. 1997. The use of bacteriophage-based systems for the separation and concentration of Salmonella . Journal of applied microbiology, 83, 259-265.
BHUSHAN, S. & ETZEL, M. R. Charged Ultrafiltration Membranes for Protein Separation. 2007.
CADEMARTIRI, R., ANANY, H., GROSS, I., BHAYANI, R., GRIFFITHS, M. & BROOK, M. A. 2010. Immobilization of bacteriophages on modified silica particles. Biomaterials, 31, 1904-1910.
CLARK, W. A. 1962. Comparison of several methods for preserving bacteriophages. Appl Microbiol, 10, 466-71.
COSTA, C. I., AZEREDO, J., BALC50, V. M., CASTRO, L. M., SANTOS, S., TEIXEIRA, J. A., MATOS, C. M., AZEVEDO, A. F. & MOUTINHO, C. M. A multiple emulsion formulation of bacteriophage encapsulated in lipid nanovesicles. MicroBiotec09, 28-30 Nov. 2009 2009 Vilamoura. 53.
COX, C. S., HARRIS, W. J. & LEE, J. 1974. Viability and Electron Microscope Studies of Phages T3 and T7 Subjected to Freeze-drying, Freeze-thawing and Aerosolization. Journal of general microbiology, 81, 207-215.
CUMMINGS, D. J., KUSY, A. R., CHAPMAN, V. A., DELONG, S. S. & STONE, K. R. 1970. Characterization of T-Even Bacteriophage Substructures: I. Tail Fibers and Tail Tubes. The Journal of Virology, 6, 534-544.
DAVIES, J. D. & KELLY, M. J. 1969. The preservation of bacteriophage H1 of Corynebacterium ulcerans U103 by freeze-drying. J Hyg (Lond), 67, 573-83.
DINI, C., ISLAN, G. A., DE URRAZA, P. J. & CASTRO, G. R. 2012. Novel Biopolymer Matrices for Microencapsulation of Phages: Enhanced Protection Against Acidity and Protease Activity. Macromolecular Bioscience, 12, 1200-1208.
EVOY, S., SINGH, A. & GLASS, N. 2009. Bacteriophage immobilization for biosensors. Ser. No. 12/466,089.
GERVAIS, L., GEL, M., ALLAIN, B., TOLBA, M., BROVKO, L., ZOUROB, M., MANDEVILLE, R., GRIFFITHS, M. & EVOY, S. 2007. Immobilization of biotinylated bacteriophages on biosensor surfaces. Sensors and Actuators B: Chemical, 125, 615-621.
GOLSHAHI, L., LYNCH, K. H., DENNIS, J. J. & FINLAY, W. H. 2011. In vitro lung delivery of bacteriophages KS4-M and φKZ using dry powder inhalers for treatment of Burkholderia cepacia complex and Pseudomonas aeruginosa infections in cystic fibrosis. Journal of applied microbiology, 110, 106-117.
HANDA, H., GURCZYNSKI, S., JACKSON, M. P., AUNER, G., WALKER, J. & MAO, G. 2008. Recognition of Salmonella Typhimurium by immobilized phage P22 monolayers. Surface Science, 602, 1392-1400.
HUGH, S. & MICHAEL, M. 2009. Immobilisation and stabilisation of virus. Ser. No. 10/512,635.
JIAYI, Z., KRAFT, B. L., YANYING, P., WALL, S. K., SAEZ, A. C. & EBNER, P. D. 2010. Development of an anti-Salmonella phage cocktail with increased host range. Foodborne Pathogens and Disease, 7, 1415-1419.
JIRKU, V. 1999. Whole cell immobilization as a means of enhancing ethanol tolerance. Journal of Industrial Microbiology and Biotechnology, 22, 147-151.
KARAM, J. K., DRAKE, J. W. & KREUZER, K. N. 1994. Molecular Biology of Bacteriophage T4, Washington, D.C., ASM.
KLEIN, J. & ZIEHR., H. 1990. Immobilization of microbial cells by adsorption. Journal of Biotechnology, 16, 1-15.
KNAEBEL, D. B., STORMO, K. E. & CRAWFORD, R. L. 1997. Immobilization of Bacteria in Macro- and Microparticles. Bioremediation Protocols.
KOREHEI, R. & KADLA, J. 2013. Incorporation of T4 bacteriophage in electrospun fibres. Journal of applied microbiology, n/a-n/a.
LAKSHMANAN, R. S., GUNTUPALLI, R., HU, J., KIM, D.-J., PETRENKO, V. A., BARBAREE, J. M. & CHIN, B. A. 2007. Phage immobilized magnetoelastic sensor for the detection of Salmonella Typhimurium. Journal of microbiological methods, 71, 55-60.
MA, Y., PACAN, J. C., WANG, Q., XU, Y., HUANG, X., KORENEVSKY, A. & SABOUR, P. M. 2008. Microencapsulation of bacteriophage Felix O1 into chitosan-alginate microspheres for oral delivery. Applied and Environmental Microbiology, 74, 4799-4805.
MILLETTE, M., LE TIEN, C., SMORAGIEWICZ, W. & LACROIX, M. 2007. Inhibition of Staphylococcus aureus on beef by nisin-containing modified alginate films and beads. Breakdowns in Food Safety, 18, 878-884.
MINIKH, 0., TOLBA, M., BROVKO, L. Y. & GRIFFITHS, M. W. 2010. Bacteriophage-based biosorbents coupled with bioluminescent ATP assay for rapid concentration and detection of Escherichia coli. Journal of microbiological methods, 82, 177-183.
MURTHY, K. & ENGELHARDT, R. 2012. Encapsulated bacteriophage formulation. Ser. No. 13/528,215.
MURTHY, K. & RAINER, E. 2008. Stabilized bacteriophage formulations. WO/2006/047870.
NANDURI, V., BALASUBRAMANIAN, S., SISTA, S., VODYANOY, V. J. & SIMONIAN, A. L. 2007a. Highly sensitive phage-based biosensor for the detection of μ-galactosidase. Analytica Chimica Acta, 589, 166-172.
NANDURI, V., BHUNIA, A. K., TU, S.-I., PAOLI, G. C. & BREWSTER, J. D. 2007b. SPR biosensor for the detection of L. monocytogenes using phage-displayed antibody. Biosensors and Bioelectronics, 23, 248-252.
NANDURI, V., SOROKULOVA, I. B., SAMOYLOV, A. M., SIMONIAN, A. L., PETRENKO, V. A. & VODYANOY, V. 2007c. Phage as a molecular recognition element in biosensors immobilized by physical adsorption. Biosensors & bioelectronics, 22, 986-992.
PAN, H., WENNA, B., ZHIPENG, X., JIANGUO, Z. & YONGQUAN, L. 2010. Immobilization of Escherichia coli cells with cis-epoxysuccinate hydrolase activity for d-tartaric acid production. Biotechnology Letters, 32, 235-241.
PASCHKE, M. 2006. Phage display systems and their applications. 70.
PASTRE, D., PIETREMENT, O., FUSIL, S., LANDOUSY, F., JEUSSET, J., DAVID, M.-O., HAMON, L., LE CAM, E. & ZOZIME, A. 2003. Adsorption of DNA to mica mediated by divalent counterions: A theoretical and experimental study. Biophysical journal, 85, 2507-2518.
PUAPERMPOONSIRI, U., FORD, S. J. & VAN DER WALLE, C. F. 2010. Stabilization of bacteriophage during freeze drying. International journal of pharmaceutics, 389, 168-175.

PUAPERMPOONSIRI, U., SPENCER, J. & VAN DER WALLE, C. F. 2009. A freeze-dried formulation of bacteriophage encapsulated in biodegradable microspheres. European Journal of Pharmaceutics and Biopharmaceutics, 72, 26-33.
SALALHA, W., KUHN, J., DROR, Y. & ZUSSMAN, E. 2006. Encapsulation of bacteria and viruses in electrospun nanofibres. Nanotechnology, 17, 4675.
SANTIAGO-SILVA, P., SOARES, N. F. F., NOBREGA, J. E., JUNIOR, M. A. W., BARBOSA, K. B. F., VOLP, A. C. P., ZERDAS, E. R. M. A. & WURLITZER, N. J. 2009. Antimicrobial efficiency of film incorporated with pediocin (ALTA® 2351) on preservation of sliced ham. Food Control, 20, 85-89.
SELVARAJ, P. T., LITTLE, M. H. & KAUFMAN, E. N. 1997. Biodesulfurization of flue gases and other sulfate/sulfite waste streams using immobilized mixed sulfate-reducing bacteria. Biotechnology progress, 13, 583-589.
SERWER, P. & HAYES, S. J. 1982. Agarose gel electrophoresis of bacteriophages and related particles. I. Avoidance of binding to the gel and recognizing of particles with packaged DNA. Electrophoresis, 3, 76-80.
SHABANI, A., ZOUROB, M., ALLAIN, B., MARQUETTE, C. A., LAWRENCE, M. F. & MANDEVILLE, R. 2008. Bacteriophage-modified microarrays for the direct impedimetric detection of bacteria. Analytical Chemistry, 80, 9475-9482.
SHAPIRA, A. & KOHN, A. 1974. The effects of freeze-drying on bacteriophage T4. Cryobiology, 11, 452-464.
SHOWE, M. K. & ONORATO, L. 1978. Kinetic factors and form determination of the head of bacteriophage T4. Proceedings of the National Academy of Sciences of the United States of America, 75, 4165-4169.
SINGH, A., GLASS, N., TOLBA, M., BROVKO, L., GRIFFITHS, M. & EVOY, S. 2009. Immobilization of bacteriophages on gold surfaces for the specific capture of pathogens. Biosensors & Bioelectronics, 24, 3645-3651.
SIRAGUSA, G. R., CUTTER, C. N. & WILLETT, J. L. 1999. Incorporation of bacteriocin in plastic retains activity and inhibits surface growth of bacteria on meat. Food Microbiology, 16, 229-235.
STANFORD, K., MCALLISTER, T. A., NIU, Y. D., STEPHENS, T. P., MAZZOCCO, A., WADDELL, T. E. & JOHNSON, R. P. 2010. Oral Delivery Systems for Encapsulated Bacteriophages Targeted at Escherichia coli 0157:H7 in Feedlot Cattle. Journal of Food Protection, 73, 1304-1312.
SUN, W. 2001. Use of bioluminescent Salmonella for assessing the efficiency of constructed phage-based biosorbent. 27.
TOLBA, M., MINIKH, O., BROVKO, L. Y., EVOY, S. & GRIFFITHS, M. W. 2010. Oriented immobilization of bacteriophages for biosensor applications. Applied and Environmental Microbiology, 76, 528-535.
VAN REIS, R., BRAKE, J. M., CHARKOUDIAN, J., BURNS, D. B. & ZYDNEY, A. L. 1999. High-performance tangential flow filtration using charged membranes. Journal of Membrane Science, 159, 133-142.
YONGSHENG, M., PAGAN, J. C., QI, W., SABOUR, P. M., XIAOQING, H. & YONGPING, X. 2012. Enhanced alginate microspheres as means of oral delivery of bacteriophage for reducing Staphylococcus aureus intestinal carriage. Food Hydrocolloids, 26, 434-440.
ZIERDT, C. H. 1988. Stabilities of lyophilized Staphylococcus aureus typing bacteriophages. Applied and Environmental Microbiology, 54, 2590.
ZOUROB, M. & RIPP, S. 2010. Bacteriophage-Based Biosensors.

1. An active phage bio-ink composition comprising one or more active lytic phages, said composition suitable for printing onto a cationic substrate to immobilize said active lytic phages on said substrate, wherein said active phage is immobilized to said substrate via interaction with the active phage structure, said structure comprising a negatively charged capsid for orientated immobilization at a surface of said cationic substrate and wherein said active phage has a positively charged tail structure that is free to capture a target bacterium

2. The composition of claim 1, wherein said composition further comprises a surfactant and a viscosity modifier, wherein said surfactant is provided in an amount of up to about 5 mM and said viscosity modifier is provided in an amount of at least 10% by weight of the composition.

3. The composition of claim 1, wherein said composition further comprises an additive selected from the group consisting of dispersants, propellants, biocides, defoamers, slip and leveling agents, plasticizers, humectants, viscosity modifiers, antioxidants, UV absorbers, tackifiers, adhesives, colorants, dyes, conductivity enhancing agents and combinations thereof.

4. The composition of claim 1, wherein said active lytic phage is provided in an amount selected from from about 10⁵ PFU/ml to about 10¹² PFU/ml, from about 10⁵ PFU/ml to about 10⁶ PFU/ml, from about 10⁷ PFU/ml, to about 10⁸ PFU/ml, from about 10⁹ PFU/ml to about 10¹¹ PFU/ml, or about 10¹² PFU/ml.

5. The composition of claim 1, wherein said active lytic phage is from the family of Levirividae, Siphoviridae or Myoviridae.

6. The composition of claim 5, wherein said active lytic phage is specific for a food borne bacteria selected from the group consisting of Campylobacterjejuni, Clostridum botulinum, Clostridium perfringens, S. enterica selected from S. Enteritidis and S. Typhimurium, Cronobacter, E. coli selected from E. coli 0157:H7 and E. coli O45:H2, Listeria monocytogenes, Vibrio and Shigella.

7. The composition of claim 5, wherein said active lytic phage is selected from the group consisting of rv5, LmoM-AG20(V20), EcoP-NAG24, Sen-AG11, SnwM-CGG4-1, Cgg 4-1, FF-47 and combinations thereof.

8. A food packaging material comprising the composition of claim 1.

9. A phage-based bioactive test strip for use in methods to detect target bacterial pathogens in a sample, the dipstick comprising a phage-based bioactive substrate comprising the composition of claim 1, wherein the phage is oriented and immobilized at the substrate surface through heads, with said phage tail fibers free to capture the target bacterial pathogens in said sample.

10. A printed cationic substrate, said substrate comprising a lytic phage bio-ink composition comprising:
one or more lytic active phages specific for food borne pathogenic bacteria;
surfactant;
viscosity modifier; and
optional conventional additives,
wherein said composition is printed on said cationic substrate such that the lytic phages are immobilized on said substrate in an orientated manner to capture a target bacteria.

11. The printed cationic substrate of claim 10, wherein said active lytic phases are orientated such that appositively charged tail structure of said phase is free to capture said food borne pathogenic bacteria.

12. The printed cationic substrate of claim 11, wherein said substrate is a cationic paper, casing, film or edible film.

13. The substrate of claim 10, wherein said bacteria is selected from the group consisting of campylobacter jejuni, clostridium botulinum clostridium perfringens, cronobacter, cyclospora, E. coli 0157:H7, E. coli O45:H2, Listeria monocytogenes, salmonella, vibrio, shigella and combinations thereof.

14. The substrate of claim 10, wherein said surfactant is provided in an amount of up to about 5 mM and is a non-ionic hydrocarbon surfactant and said viscosity modifier is provided in an amount of at least 10% by weight of the composition.

15. The substrate of claim 10, wherein said active lytic phage is provided in an amount selected from about 10⁵ PFU/ml to about 10¹² PFU/ml, from about 10⁵ PFU/ml to about 10⁶ PFU/ml, from about 10′ PFU/ml, to about 10⁸ PFU/ml, from about 10⁹ PFU/ml to about 10¹¹ PFU/ml, or about 10¹² PFU/ml per cm² of substrate.

16. The substrate of claim 10, wherein said active lytic phage is from the family of Levirividae, Siphoviridae or Myoviridae and is specific for a food borne bacteria selected from the group consisting of Campylobacterjejuni, Clostridum botulinum, Clostridium perfringens, S. enterica selected from S. Enteritidis and S. Typhimurium., Cronobacter, E. coli selected from E. coli 0157:H7 and E. coli O45:H2, Listeria monocytogenes,Vibrio and Shigella.

17. The substrate of claim 16, wherein said active lytic phage is selected from the group consisting of rv5, LmoM-AG20(V20), EcoP-NAG24, Sen-AG 11, SnwM-CGG4-1, Cgg 4-1, FF-47 and combinations thereof.

18. A food packaging material comprising the substrate of claim 12.

19. A bioactive test dip stick for detect bacterial pathogens, the dip stick comprising the substrate of claim 10.

20. A method to make a phage-based bioactive paper, the method comprising:
printing an active lytic phage bio-ink composition comprising one or more lytic phages specific for food borne pathogenic bacteria, surfactant and viscosity modifier onto a cationic paper; wherein said printing is done by a piezoelectric ink jet printer,
drying the printed cationic paper and maintaining the printed cationic paper at a relative humidity of about 80% to 85%, wherein about 10⁶ PFU of phage is provided per cm² of paper.
